(19) United States
(12) Patent Application Publication
(10) Pub. No.: US 2023/0322735 A1
(43) Pub. Date: Oct. 12, 2023

(54) AZETIDINYL TRYPTAMINES AND METHODS OF TREATING PSYCHIATRIC DISORDERS

(71) Applicant: GILGAMESH PHARMACEUTICALS, INC., New York, NY (US)

(72) Inventor: Andrew Carry KRUEGEL, New York, NY (US)

(73) Assignee: GILGAMESH PHARMACEUTICALS, INC., New York, NY (US)

(21) Appl. No.: 18/024,545

(22) PCT Filed: Sep. 3, 2021
(86) PCT No.: PCT/US2021/049149

§ 371 Date: Mar. 3, 2023

Related U.S. Application Data
(60) Provisional application No. 63/074,557, filed on Sep. 4, 2020.

(51) Int. Cl. C07D 403/06; C07F 9/572; A61P 25/24

(52) U.S. Cl. CPC C07D 403/06; C07F 9/5728; A61P 25/24

(57) ABSTRACT

The present disclosure includes azetidinyl tryptamines and methods of treating psychiatric disorders with such compounds. Also provided are pharmaceutical compositions that include azetidinyl tryptamines.

### TECHNICAL FIELD

Novel azetidinyl tryptamines and methods of treating psychiatric disorders with such compounds. Also provided are pharmaceutical compositions that include azetidinyl tryptamines.

### BACKGROUND

Tryptamines are a diverse class of alkaloids containing the structural scaffold of the natural alkaloid tryptamine.

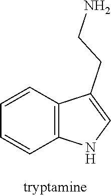 [SMILES: NCCc1c[nH]c2ccccc12]

There are a significant number of tryptamine compounds that include naturally occurring compounds, as well as synthetic and semi-synthetic chemical derivatives with similar structure. Tryptamines are known to have diverse psychoactive and physiological effects. Some tryptamines are serotonin 2A (5-HT2A) receptor agonists and/or modulators of other serotonin receptors and are known to be psychoactive and/or induce vasoconstriction. In some cases, such compounds induce prolonged hallucinations. Other tryptamines are modulators of monoamine transporters. The most well-known tryptamines are psychedelic compounds, including compounds derived from entheogenic fungi (psilocybin and psilocin), DMT, LSD, 5-MeO-DMT, bufotenin, and ibogaine. These compounds are known to have significant effects on thought, perception, and behavior. However, these compounds are currently classified as Schedule I drugs under the Controlled Substances Act due to their high abuse potential, no accepted medical use, and lack of established safety. Moreover, tryptamines are metabolized by a number of pathways, in some cases including monoamine oxidase, limiting the oral bioavailability of some compounds.

Accordingly, there remains a need for safe and effective tryptamine compounds that can reliably be used for the treatment of psychiatric disorders.

### SUMMARY

The present disclosure provides a compound having the general Formula I:

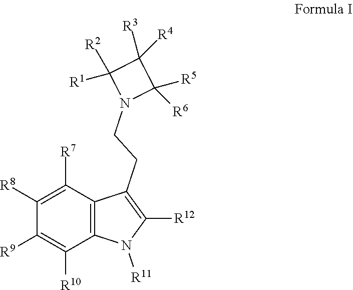

wherein
R¹-R⁶ are each independently selected from the group consisting of H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, and C₁-C₅ halo-alkyl;
R⁷-R¹⁰ and R¹² are each independently selected from the group consisting of H, F, Cl, Br, I, CF₃, SF₅, C₁-C₁₀ alkyl, C₂-C₁₀ alkenyl, C₂-C₁₀ alkynyl, C₁-C₁₀ heteroalkyl, C₂-C₁₀ heteroalkenyl, C₂-C₁₀ heteroalkynyl, C₁-C₁₀ halo-alkyl, —CN, —O—(C₁-C₁₀ alkyl), —O—(C₁-C₁₀ heteroalkyl), —S—(C₁-C₁₀ alkyl), —S—(C₁-C₁₀ heteroalkyl), —S(O)—(C₁-C₁₀ alkyl), —SO₂—(C₁-C₁₀ alkyl), OH, —CO₂H, —C(O)—NH₂, —C(O)—NH—(C₁-C₁₀ alkyl), —CO₂—(C₁-C₁₀ alkyl), —O—C(O)—(C₁-C₁₀ alkyl), —O—P(O)(OH)(OH), NH₂, —NH—(C₁-C₁₀ alkyl), —N(C₁-C₁₀ alkyl)(C₁-C₁₀ alkyl), NO₂, and OCF₃; and
R¹¹ is selected from the group consisting of H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, and C₁-C₅ halo-alkyl;
   or a pharmaceutically acceptable salt or ester thereof.

The present disclosure further provides a pharmaceutical composition comprising one or more compounds of the present disclosure.

The present disclosure further provides a method of treating a psychiatric disease or disorder in a patient in need thereof, said method comprising administering to said subject a composition comprising an effective amount of a compound of the present disclosure.

### BRIEF DESCRIPTION OF THE DRAWINGS

FIG. 1. Immobility time in the FST. A one-way ANOVA revealed a significant main effect of treatment (F(5,54)=19.35, P<0.0001) on the total time spent immobile in the FST. Dunnett's multiple comparisons test was used to test if a group was significantly different from vehicle. All treatments were significantly different from vehicle. **** P<0.0001 vs. vehicle.

FIG. 2. Swimming time in the FST. A one-way ANOVA revealed a significant main effect of treatment (F(5,54)=9.606, P<0.0001) on the total time spent swimming in the FST. Dunnett's multiple comparisons test was used to test if a group was significantly different from vehicle. ** P<0.01, **** P<0.0001 vs. vehicle.

### DETAILED DESCRIPTION

In the following detailed description, numerous specific details are set forth in order to provide a thorough understanding of the present disclosure. However, it will be understood by those skilled in the art that the present disclosure may be practiced without these specific details. In other instances, well-known methods, procedures, and components have not been described in detail so as not to obscure the present disclosure.

Described herein are novel azetidinyl tryptamines and methods of treating psychiatric disorders with such compounds. Also provided are pharmaceutical compositions that include azetidinyl tryptamines. The compounds provided have greater potency as serotonin 1A (5-HT1A) receptor agonists compared to their acyclic counterparts, such as tryptamines bearing an N,N-dimethyl substituent. Further, they have better metabolic stability than such N,N-dimethyl counterparts.

The present disclosure provides a compound having the general Formula I:

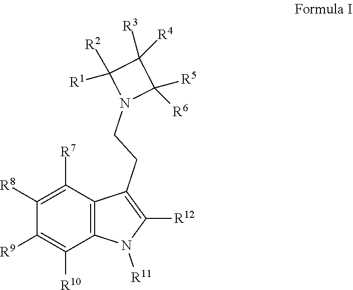

### Wherein

R¹-R⁶ are each independently selected from the group consisting of —H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, and C₁-C₅ halo-alkyl;
R⁷-R¹⁰ and R¹² are each independently selected from the group consisting of —H, —F, —Cl, —Br, —I, —CF₃, —SF₅, C₁-C₁₀ alkyl, C₂-C₁₀ alkenyl, C₂-C₁₀ alkynyl, C₁-C₁₀ heteroalkyl, C₂-C₁₀ heteroalkenyl, C₂-C₁₀ heteroalkynyl, C₁-C₁₀ halo-alkyl, —CN, —O—(C₁-C₁₀ alkyl), —O—(C₁-C₁₀ heteroalkyl), —S—(C₁-C₁₀ alkyl), —S—(C₁-C₁₀ heteroalkyl), —S(O)—(C₁-C₁₀ alkyl), —SO₂—(C₁-C₁₀ alkyl), OH, —CO₂H, —C(O)—NH₂, —C(O)—NH—(C₁-C₁₀ alkyl), —CO₂—(C₁-C₁₀ alkyl), —O—C(O)—(C₁-C₁₀ alkyl), —O—P(O)(OH)(OH), NH₂, —NH—(C₁-C₁₀ alkyl), —N(C₁-C₁₀ alkyl)(C₁-C₁₀ alkyl), —NO₂, and —OCF₃; and
R¹¹ is selected from the group consisting of —H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, C₁-C₅ halo-alkyl;
   or a pharmaceutically acceptable salt or ester thereof.

In embodiments,
R¹-R⁶ are each independently selected from the group consisting of —H, -Me, -Et, -n-Pr, -i-Pr, cyclopropyl, —CH═CH₂ (vinyl), —C≡CH (ethynyl), and —CH₂CHCH₂ (allyl);
R⁷-R¹⁰ and R¹² are each independently selected from the group consisting of —H, —F, —Cl, —Br, —I, —CF₃, —SF₅, -Me, -Et, -n-Pr, -i-Pr, cyclopropyl, —CH═CH₂ (vinyl), —C≡CH (ethynyl), —CH₂CHCH₂ (allyl), —CN, —OMe, —OEt, —SMe, —SEt, —OH, —OAc, —CO₂H, —C(O)—NH₂, —CO₂Me, —O—C(O)—(C₁-C₅ alkyl), —O—P(O)(OH)(OH), —NH₂, —NO₂, and —OCF₃; and
R¹¹ is selected from the group consisting of —H, -Me, -Et, -n-Pr, -i-Pr, cyclopropyl, and —CH₂CHCH₂ (allyl);
   or a pharmaceutically acceptable salt or ester thereof.

In embodiments,
R¹-R⁶ are each independently selected from the group consisting of —H, -Me, and -Et;
R⁷-R¹⁰ and R¹² are each independently selected from the group consisting of —H, —F, —Cl, —Br, —I, —CF₃, —SF₃, -Me, -Et, —CN, —OMe, —SMe, —OH, —OAc, —C(O)—NH₂, —O—P(O)(OH)(OH), —NH₂, —NO₂, and —OCF₃; and
R¹¹ is selected from the group consisting of —H, -Me, and -Et;
   or a pharmaceutically acceptable salt or ester thereof.

In embodiments,
R¹-R⁶ are each independently selected from the group consisting of —H, -Me, and -Et:
R⁷-R¹⁰ and R¹² are each independently selected from the group consisting of —H, —F, —Cl, —Br, —I, —CF₃, -Me, —CN, —OMe, —OH, —OAc, —C(O)—NH₂, —O—P(O)(OH)(OH), and —NH₂; and
R¹¹ is —H,
   or a pharmaceutically acceptable salt or ester thereof.

In some embodiments, a compound is represented by Formula (I-a):

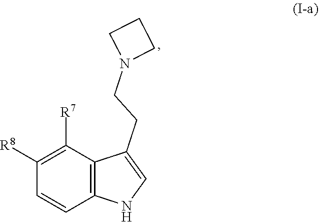

or a pharmaceutically acceptable salt thereof.

In some embodiments, R⁷ is selected from the group consisting of —H, —OH, —O—(C₁-C₁₀ alkyl), —O—C(O)—(C₁-C₁₀ alkyl), and —O—P(O)(OH)(OH). In some embodiments, R⁷ is selected from the group consisting of —H, —OH, —OAc, and —O—P(O)(OH)(OH). In some embodiments, R⁸ is selected from the group consisting of —H, —OH, —O—(C₁-C₁₀ alkyl), and —O—C(O)—(C₁-C₁₀ alkyl). In some embodiments, R⁸ is selected from the group consisting of —H, —OH, —OMe and —OAc.

In embodiments, a compound of the present disclosure is selected from:

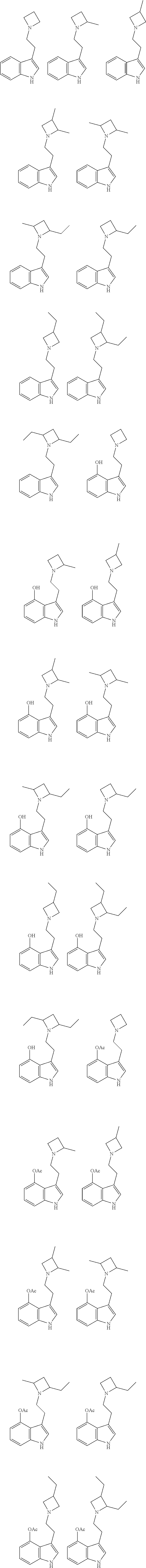

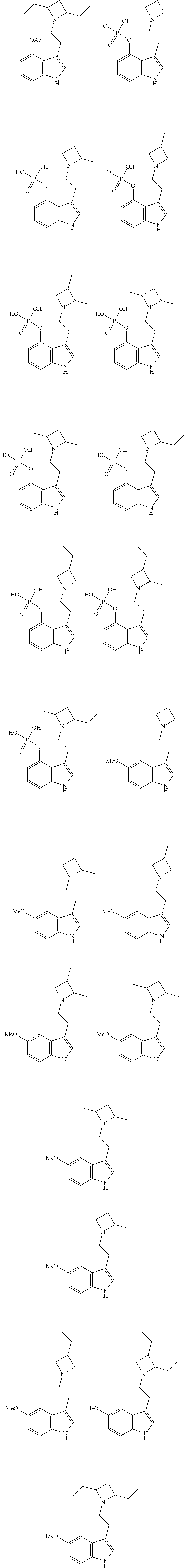

or a pharmaceutically acceptable salt or ester thereof.

In embodiments, a compound of the present disclosure is selected from:

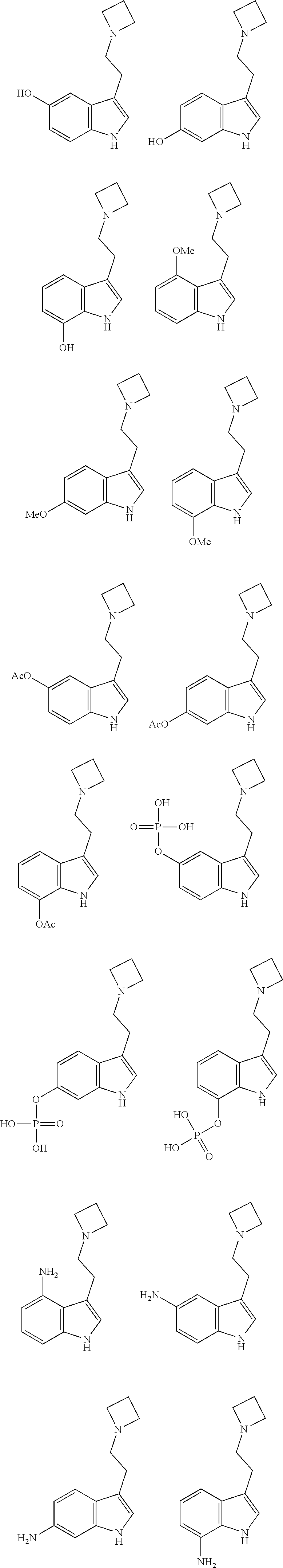

or a pharmaceutically acceptable salt or ester thereof.

In embodiments, a compound of the present disclosure is selected from:

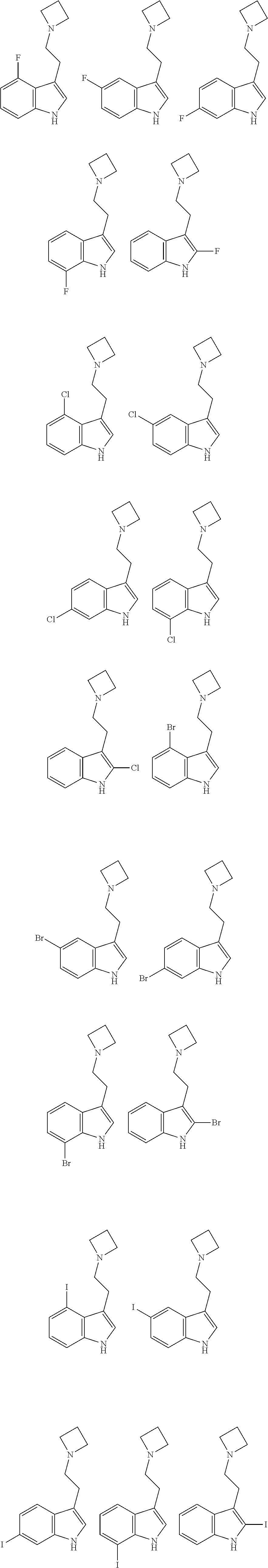

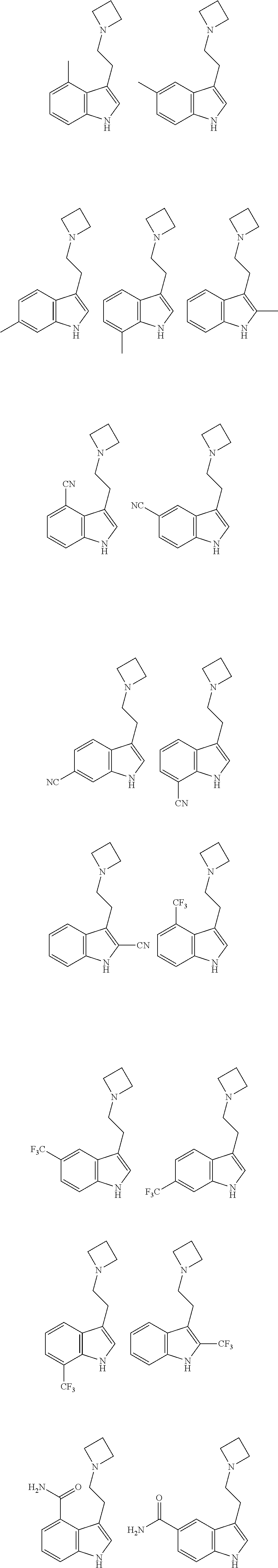

or a pharmaceutically acceptable salt thereof.

In embodiments, a compound of the present disclosure is selected from:

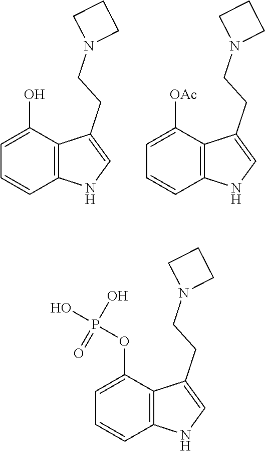

or a pharmaceutically acceptable salt or ester thereof.

In embodiments, the compound of the present disclosure has the structure:

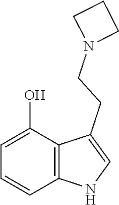 [SMILES: Oc1cccc2[nH]cc(CCN3CCC3)c12]

or a pharmaceutically acceptable salt thereof.

In embodiments, the compound of the present disclosure has the structure:

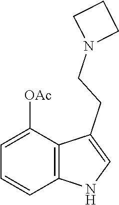 [SMILES: CC(=O)Oc1cccc2[nH]cc(CCN3CCC3)c12]

or a pharmaceutically acceptable salt thereof.

In embodiments, the compound of the present disclosure has the structure:

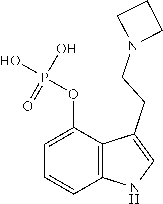 [SMILES: O=P(O)(O)Oc1cccc2[nH]cc(CCN3CCC3)c12]

or a pharmaceutically acceptable salt thereof.

In embodiments, the compound of the present disclosure has the structure:

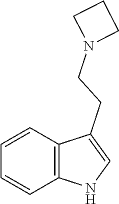 [SMILES: c1ccc2c(CCN3CCC3)c[nH]c2c1]

or a pharmaceutically acceptable salt thereof.

In embodiments, the compound of the present disclosure has the structure:

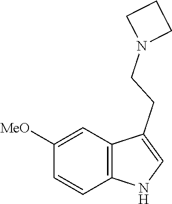 [SMILES: COc1ccc2[nH]cc(CCN3CCC3)c2c1]

or a pharmaceutically acceptable salt thereof.

The present disclosure further provides a pharmaceutical composition comprising one or more compounds of the present disclosure.

The present disclosure further provides a method of treating a psychiatric disease or disorder in a patient in need thereof, said method comprising administering to said subject a composition comprising an effective amount of a compound of the present disclosure.

In embodiments, the psychiatric disease or disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

In embodiments, the psychiatric disease or disorder is selected from the group consisting of bipolar disorder I, bipolar disorder II, cyclothymic disorder, substance/medication-induced bipolar and related disorder, and bipolar and related disorder due to another medical condition.

In embodiments, the psychiatric disease or disorder is a substance-related disorder or substance-use disorder.

In embodiments, the psychiatric disease or disorder is selected from the group consisting of separation anxiety disorder, selective mutism, specific phobia, social anxiety disorder, panic disorder, panic attach, agoraphobia, generalized anxiety disorder, substance/medication-induced anxiety disorder, anxiety disorder due to another medical condition.

In embodiments, the psychiatric disease or disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

In embodiments, the psychiatric disorder is a neurocognitive disorder.

In embodiments, the psychiatric disease or disorder is a treatment-resistant disease or disorder.

In embodiments, the method provides improvement in at least one symptom selected from the group consisting of sadness or lethargy or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, irritability, fatigue, feelings of worthlessness or low self-esteem, strongly held negative beliefs or pessimistic thoughts about self, others or world, feelings of helplessness, inability to concentrate or distractibility, recurrent thoughts of death or suicide, feelings of guilt, memory complaints, difficulty experiencing positive feelings, feeling cut off or distant from people, hypervigilance, risk taking behavior, avoidance of thoughts about a stressful or traumatic event, pains and aches, ruminations and obsessive thoughts, compulsive behaviors, talking to people you don't know well or strangers, being center of attention, disturbing intrusive thoughts, can't get through week without drug use, guilty about drug use, problems with friends or family due to drug use, and withdrawal symptoms due to drug use.

The present disclosure further provides a method of enhancing creativity or cognition in a subject, said method comprising administering to said subject a composition comprising an effective amount of a compound of the present disclosure.

In embodiments, the methods and compositions may be used to treat a psychiatric disorder including Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, Depressive Disorder Due to Another Medical Condition.

Also provided herein are methods of treating refractory depression, e.g., patients suffering from a depressive disorder that does not, and/or has not, responded to adequate courses of at least one, or at least two, other antidepressant compounds or therapeutics. As used herein “depressive disorder” encompasses refractory depression.

In embodiments, the methods and compositions may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, Bipolar and Related Disorder Due to Another Medical Condition.

In embodiments, the methods and compositions may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine and tobacco. For example, the methods and compositions may be used to facilitate smoking cessation or cessation of opioid use.

In embodiments, the methods and compositions may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition.

In embodiments, the methods and compositions may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive and Related Disorder, Obsessive-Compulsive and Related Disorder Due to Another Medical Condition.

In embodiments, the methods and compositions may be used to treat a psychiatric disorder including Trauma- and Stressor-Related Disorders, e.g., Reactive Attachment Disorder, Disinhibited Social Engagement Disorder, Posttraumatic Stress Disorder, Acute Stress Disorder, and Adjustment Disorders.

In embodiments, the methods and compositions may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

In embodiments, the methods and compositions may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies.

In embodiments, the methods and compositions may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder.

In embodiments, the methods and compositions may be used to treat a psychiatric disorder including Personality Disorders, e.g., Borderline Personality Disorder.

In embodiments, the methods and compositions may be used to treat a psychiatric disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction.

In embodiments, the methods and compositions may be used to treat a psychiatric disorder including Gender Dysphoria, e.g., Gender Dysphoria.

In other embodiments provided are methods and compositions for treating migraine or cluster headache by administering to a patient in need thereof a compound of the present disclosure.

In embodiments, the terms “effective amount” or “therapeutically effective amount” refer to an amount of a compound, material, composition, medicament, or other material that is effective to achieve a particular pharmacological and/or physiologic effect including but not limited to reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide, or to provide a desired pharmacologic and/or physiologic effect, for example, reducing, inhibiting, or reversing one or more of the underlying pathophysiological mechanisms underlying neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof. The precise dosage will vary according to a variety of factors such as subject-dependent variables (e.g., age, immune system health, clinical symptoms etc.), the disease or disorder being treated, as well as the route of administration and the pharmacokinetics of the agent being administered.

In embodiments, methods include treating a psychiatric disorder, e.g., a depressive disorder, by administering to a patient in need thereof a pharmaceutical composition including about 0.01 mg to about 400 mg of a compound of the present disclosure. In embodiments, doses may be, e.g., in the range of about 0.01 to 400 mg, 0.01 to 300 mg, 0.01 to 250 mg, 0.01 to 200 mg, 0.01 to 150 mg, 0.01 to 100 mg, 0.01 to 75 mg, 0.01 to 50 mg, 0.01 to 25 mg, 0.01 to 20 mg, 0.01 to 15 mg, 0.01 to 10 mg, 0.01 to 5 mg, 0.01 to 1 mg, 0.01 to 0.5 mg, 0.01 to 0.1 mg, 0.1 to 400 mg, 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 400 mg, 10 to 300 mg, 10 to 250 mg, 10 to 200 mg, 10 to 150 mg, 10 to 100 mg, 10 to 50 mg, 10 to 25 mg, 10 to 15 mg, 20 to 400 mg, 20 to 300 mg, 20 to 250 mg, 20 to 200 mg, 20 to 150 mg, 20 to 100 mg, 20 to 50 mg, 50 to 400 mg, 50 to 300 mg, 50 to 250 mg, 50 to 200 mg, 50 to 150 mg, 50 to 100 mg, 100 to 400 mg, 100 to 300 mg, 100 to 250 mg, 100 to 200 mg, with doses of, e.g., about 0.01 mg, 0.025 mg, 0.05 mg, 0.1 mg, 0.15 mg, 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In specific embodiments, dosages may include amounts of a compound of the present disclosure present disclosure or a pharmaceutically acceptable salt thereof in the range of about, e.g., 1 mg to 50 mg, 1 mg to 40 mg, 1 mg to 30 mg, 1 mg to 20 mg, 1 mg to 15 mg, 1 mg to 10 mg, 0.1 mg to 10 mg, 0.1 to 5 mg, or 0.1 to 1 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12.5 mg, 15 mg, 17.5 mg, 20 mg, 22.5 mg, 25 mg, 27.5 mg, 30 mg, 35 mg, 40 mg, 45 mg, and 50 mg being specific examples of doses.

Typically, dosages of a compound of the present disclosure or a pharmaceutically acceptable salt thereof, are administered once, twice, three or four times daily, every other day, every three days, twice weekly, once weekly, twice monthly, or once monthly to a patient in need thereof. In embodiments, the dosage is about, e.g., 0.1-400 mg/day, 0.1-300 mg/day, 0.1-250 mg/day, 0.1-200 mg/day, 0.1-100 mg/day, 0.1-50 mg/day, or 0.1 to 25 mg/day, for example 300 mg/day, 250 mg/day, 200 mg/day, 150 mg/day, 100 mg/day, 75 mg/day, 50 mg/day, 25 mg/day, 20 mg/day, 10 mg/day, 5 mg/day, 2.5 mg/day, 1 mg/day, 0.5 mg/day, 0.25 mg/day, or 0.1 mg/day. In embodiments, the foregoing example dose ranges may be delivered over intervals longer than one day, e.g. 0.1-400 mg/week.

In embodiments, pharmaceutical compositions for parenteral or inhalation, e.g., a spray or mist, administration of a compound of the present disclosure or a pharmaceutically acceptable salt thereof, having a concentration of about 0.005 mg/mL to about 500 mg/mL. In embodiments, the compositions include a compound of the present disclosure or a pharmaceutically acceptable salt thereof, at a concentration of, e.g., about 5 mg/mL to about 500 mg/mL, about 5 mg/mL to about 100 mg/mL, about 5 mg/mL to about 50 mg/mL, about 1 mg/mL to about 100 mg/mL, about 1 mg/mL to about 50 mg/mL, about 0.1 mg/mL to about 25 mg/mL, about 0.1 mg/mL to about 10 mg/mL, about 0.05 mg/mL to about 10 mg/mL, about 0.05 mg/mL to about 5 mg/mL, about 0.05 mg/mL to about 1 mg/mL, about 0.005 mg/mL to about 1 mg/mL, about 0.005 mg/mL to about 0.25 mg/mL, or about 0.005 mg/mL to about 0.1 mg/mL.

In embodiments, the composition includes a compound of the present disclosure or a pharmaceutically acceptable salt thereof, at a concentration of, e.g., about 0.05 mg/mL to about 500 mg/mL, about 0.05 mg/mL to about 100 mg/mL, about 0.05 mg/mL to about 50 mg/mL, about 0.05 mg/mL to about 25 mg/mL, about 0.05 mg/mL to about 10 mg/mL, about 0.05 mg/mL to about 5 mg/mL, about 0.005 mg/mL to about 1 mg/mL, about 0.005 mg/mL to about 0.25 mg/mL, about 0.005 mg/mL to about 0.05 mg/mL, or about 0.005 mg/mL to about 0.025 mg/mL. In embodiments, the pharmaceutical compositions are formulated as a total volume of about, e.g., 0.1 mL, 0.25 mL, 0.5 mL, 1 mL, 2 mL, 5 mL, 10 mL, 20 mL, 25 mL, 50 mL, 100 mL, 200 mL, 250 mL, or 500 mL.

Typically, dosages may be administered to a subject once, twice, three times or four times daily, every other day, every three days, twice weekly, once weekly, twice monthly, once monthly, thrice yearly, twice yearly, or once yearly. In embodiments, a compound of the present disclosure or a pharmaceutically acceptable salt thereof) is administered to a subject once in the morning, or once in the evening. In embodiments, a compound of the present disclosure or a pharmaceutically acceptable salt thereof is administered to a subject once in the morning, and once in the evening. In embodiments, a compound of the present disclosure or a pharmaceutically acceptable salt thereof is administered to a subject three times a day (e.g., at breakfast, lunch, and dinner), at a dose, e.g., of 0.5 mg/administration (e.g., 1.5 mg/day).

In embodiments, a compound of the present disclosure or a pharmaceutically acceptable salt thereof is administered to a subject at a dose of 0.5 mg/day in one or more doses. In embodiments, a compound of the present disclosure or a pharmaceutically acceptable salt thereof is administered to a subject at a dose of 1 mg/day in one or more doses. In embodiments, a compound of the present disclosure or a pharmaceutically acceptable salt thereof is administered to a subject at a dose of 2.5 mg/day in one or more doses. In embodiments, a compound of the present disclosure or a pharmaceutically acceptable salt thereof is administered to a subject at a dose of 5 mg/day in one or more doses. In embodiments, a compound of the present disclosure or a pharmaceutically acceptable salt thereof is administered to a subject at a dose of 10 mg/day in one or more doses. In embodiments, a compound of the present disclosure or a pharmaceutically acceptable salt thereof is administered to a subject at a dose of 15 mg/day in one or more doses. In embodiments, a compound of the present disclosure or a pharmaceutically acceptable salt thereof is administered to a subject at a dose of 20 mg/day in one or more doses. In embodiments, a compound of the present disclosure or a pharmaceutically acceptable salt thereof is administered to a subject at a dose of 25 mg/day in one or more doses. In embodiments, a compound of the present disclosure or a pharmaceutically acceptable salt thereof is administered to a subject at a dose of 30 mg/day in one or more doses. In embodiments, a compound of the present disclosure or a pharmaceutically acceptable salt thereof is administered to a subject at a dose of 40 mg/day in one or more doses. In embodiments, a compound of the present disclosure or a pharmaceutically acceptable salt thereof is administered to a subject at a dose of 50 mg/day in one or more doses.

In embodiments, the dosage of a compound of the present disclosure or a pharmaceutically acceptable salt thereof is 0.0005-5 mg/kg, 0.001-1 mg/kg, 0.01-1 mg/kg or 0.1-5 mg/kg once, twice, three times or four times daily. For example, in embodiments, the dosage is 0.0005 mg/kg, 0.001 mg/kg, 0.005 mg/kg, 0.01 mg/kg, 0.025 mg/kg, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 1 mg/kg, 2.5 mg/kg, 5 mg/kg, once, twice, three times or four times daily. In embodiments, a subject is administered a total daily dose of 0.01 mg to 500 mg of a compound of the present disclosure or a pharmaceutically acceptable salt thereof once, twice, three times, or four times daily. In embodiments, the total amount administered to a subject in 24-hour period is, e.g., 0.01 mg, 0.025 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 12.5 mg, 15 mg, 17.5 mg 20 mg, 25 mg, 30 mg, 35 mg, 40 mg, 50 mg, 60 mg, 75 mg, 100 mg, 150 mg, 200 mg, 300 mg, 400 mg, 500 mg. In embodiments, the subject may be started at a low dose and the dosage is escalated. In embodiments, the subject may be started at a high dose and the dosage is decreased.

In embodiments, a compound of the present disclosure or a pharmaceutically acceptable salt thereof is administered to a patient under the supervision of a healthcare provider.

In embodiments, a compound of the present disclosure or a pharmaceutically acceptable salt thereof is administered to a patient under the supervision of a healthcare provider at a clinic specializing in the delivery of psychoactive treatments.

In embodiments, a compound of the present disclosure is administered to a patient under the supervision of a healthcare provider at a high dose intended to induce a psychedelic experience in the subject, e.g., 5 mg, 7.5 mg, 10 mg, 12.5 mg, 15 mg, 17.5 mg, 20 mg, 25 mg, 30 mg, 35 mg, 40 mg, 50 mg, 60 mg, 70 mg, 80 mg, 90 mg, or 100 mg.

In embodiments, the administration to a patient of a high dose under the supervision of a healthcare provider occurs periodically in order to maintain a therapeutic effect in the patient, e.g., every three days, twice weekly, once weekly, twice monthly, once monthly, four times yearly, thrice yearly, twice yearly, or once yearly.

In embodiments, a compound of the present disclosure or a pharmaceutically acceptable salt thereof is administered by a patient on their own at home or otherwise away from the supervision of a healthcare provider.

In embodiments, a compound of the present disclosure or a pharmaceutically acceptable salt thereof is administered by a patient on their own at home or otherwise away from the supervision of a healthcare provider at a low dose intended to be sub-perceptual or to induce threshold psychoactive effects, e.g., 0.1 mg, 0.25 mg, 0.5 mg, 0.75 mg, 1 mg, 1.5 mg, 2 mg, 2.5 mg, 3 mg, or 4 mg.

In embodiments, the administration by a patient of a low dose on their own occurs periodically in order to maintain a therapeutic effect in the patient, e.g., daily, every other day, every three days, twice weekly, once weekly, twice monthly, or once monthly,

In embodiments, a compound of the present disclosure or a pharmaceutically acceptable salt thereof may be administered, e.g., via inhalation or orally, at specified intervals. For example, during treatment a patient may be administered a compound of the present disclosure at intervals of every, e.g., 1 year, 6 months, 4 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

Suitable dosage forms for a compound of the present disclosure or a pharmaceutically acceptable salt thereof include, but are not limited to, oral forms, such as tablets, hard or soft gelatin capsules, powders, granules and oral solutions, syrups or suspensions, troches, as well as sublingual, buccal, intratracheal, intraocular, or intranasal forms, forms adapted to inhalation, topical forms, transdermal forms, or parenteral forms, for example, forms adapted for intravenous, intra-arterial, intraperitoneal, intrathecal, intraventricular, intramuscular or subcutaneous administration. In embodiments, for such parenteral administration, the pharmaceutical composition may be in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Pharmaceutical compositions herein may be provided with immediate release, delayed release, extended release, or modified release profiles. In embodiments, pharmaceutical compositions with different drug release profiles may be combined to create a two-phase or three-phase release profile. For example, pharmaceutical compositions may be provided with an immediate release and an extended-release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, glidants, disintegrants, fillers, and coating compositions.

As used herein, the term “pharmaceutically acceptable” refers to molecular entities and compositions that are “generally regarded as safe”, e.g., that are physiologically tolerable and do not typically produce an allergic or similar untoward reaction when administered to a human. In embodiments, this term refers to molecular entities and compositions approved by a regulatory agency of the federal or a state government, as the GRAS list under sections 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As used herein, the term “pharmaceutically acceptable salts” includes acid addition salts, addition salts of free bases, wherein the compound is modified by making acid or base salts thereof. Examples of pharmaceutically acceptable salts include but are not limited to mineral or organic acid salts of basic residues such as amines, and alkali or organic salts of acidic residues such as carboxylic acids. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, maleic, tolunesulfonic, naphthalenesulfonic, methanesulfonic, ethane disulfonic, and oxalic acids. The pharmaceutically acceptable salts of a compound of the present disclosure can be synthesized from the parent compound, which contains a basic or acidic moiety, by conventional chemical methods.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

In the context of the present disclosure the term “5-HT2A receptor agonist” is intended to mean any compound or substance that activates the 5-HT2A receptor. The agonist may be a partial or full agonist.

In the context of the present disclosure the term “5-HT1A receptor agonist” is intended to mean any compound or substance that activates the 5-HT1A receptor. The agonist may be a partial or full agonist.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the present disclosure or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

Tablets may contain the active ingredient compounds and suitable binders, lubricants, disintegrating agents, coloring agents, flavoring agents, flow-inducing agents, and melting agents. Gelatin capsules may contain the active ingredient compounds and powdered carriers, such as lactose, starch, cellulose derivatives, magnesium stearate, stearic acid, and the like. Similar diluents can be used to make compressed tablets. Compressed tablets can be sugar coated or film coated to mask any unpleasant taste and protect the tablet from the atmosphere, or enteric coated for selective disintegration in the gastrointestinal tract. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration can contain coloring and flavoring to increase patient acceptance.

For parenteral administration, suitable compositions include aqueous and non-aqueous sterile solutions. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration e.g. by nasal inhalation, include fine dusts or mists which may be generated by means of metered dose pressurized aerosols, nebulizers or insufflators. Parenteral and intravenous forms may also include minerals and other materials to make them compatible with the type of injection or delivery system chosen.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as prodrugs. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Pharmaceutical compositions herein may be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended-release profile. In some embodiments, pharmaceutical compositions may be provided with an extended release and delayed release profile. Such composition may be provided as pulsatile formulations, multilayer tablets, or capsules containing tablets, beads, granules, etc.

Pharmaceutical compositions herein may be provided with abuse deterrent features by techniques know in the art, for example, by making a tablet that is difficult to crush or to dissolve in water.

The present disclosure further includes a pharmaceutical composition, as hereinbefore described, in combination with packaging material, including instructions for the use of the composition for a use as hereinbefore described.

The exact dose and regimen of administration of the composition will necessarily be dependent upon the type and magnitude of the therapeutic or nutritional effect to be achieved and may vary depending on factors such as the particular compound, formula, route of administration, or age and condition of the individual subject to whom the composition is to be administered.

The compounds used in the method of the present disclosure may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

In embodiments, deuterium-enriched azetidinyl tryptamines and their use are contemplated and within the scope of the methods and compositions described herein. Deuterium can be incorporated in any position in place of hydrogen (protium) synthetically, according to synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art. Exemplary deuterium-enriched azetidinyl tryptamines include:

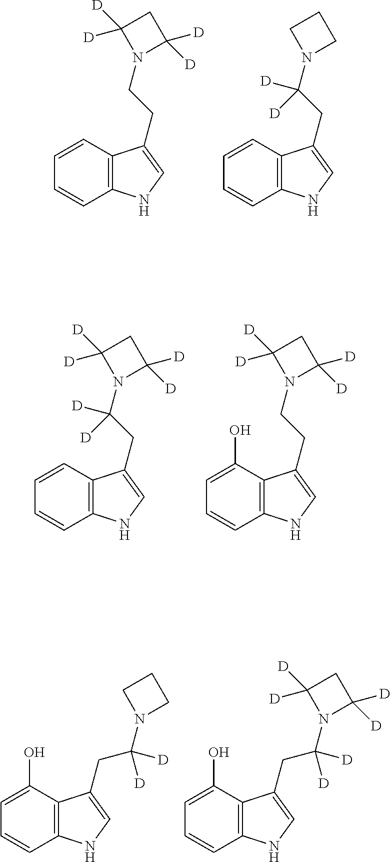 [SMILES: [2H]C1([2H])CC([2H])([2H])N1CCc1c[nH]c2ccccc12 ; [2H]C([2H])(Cc1c[nH]c2ccccc12)N1CCC1 ; [2H]C([2H])(Cc1c[nH]c2ccccc12)N1C([2H])([2H])CC1([2H])[2H] ; [2H]C1([2H])CC([2H])([2H])N1CCc1c[nH]c2cccc(O)c12 ; [2H]C([2H])(Cc1c[nH]c2cccc(O)c12)N1CCC1 ; [2H]C([2H])(Cc1c[nH]c2cccc(O)c12)N1C([2H])([2H])CC1([2H])[2H]]

or a pharmaceutically acceptable salt thereof, wherein D represents a deuterium-enriched —H site.

In embodiments, each D represents a deuterium-enriched —H site and the level of deuterium at each deuterium-enriched —H site of the compound is 0.02% to 100%.

In embodiments, each D represents a deuterium-enriched —H site and the level of deuterium at each deuterium-enriched —H site of the compound is 50%-100%, 70%-100%, 90%-100%, 95%-100%, 96%-100%, 97%-100%, 98%-100%, or 99%-100%.

The tryptamines may be racemic and/or optically active isomers thereof. In this regard, some of the compounds can have asymmetric carbon atoms, and therefore, can exist either as racemic mixtures or as individual optical isomers (enantiomers). Compounds described herein that contain a chiral center include all possible stereoisomers of the compound, including compositions including the racemic mixture of the two enantiomers, scalemic mixtures of the two enantiomers, or mixtures including each enantiomer individually, substantially free of the other enantiomer. Thus, for example, contemplated herein is a composition including the S enantiomer of a compound substantially free of the R enantiomer, or the R enantiomer substantially free of the S enantiomer. If the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than 25%, 15%, 10%, 8%, 5%, 3%, or less than 1% of the minor enantiomer or diastereomer(s).

Methods for synthesizing, isolating, preparing, and administering various stereoisomers are known in the art. Separation of diastereomers or cis and trans isomers may be achieved by conventional techniques, such as, for example, by fractional crystallization, chromatography or High Performance Liquid Chromatography (HPLC) of a stereoisomeric mixture of the agent or a suitable salt or derivative thereof. An individual enantiomer of a compound of a compound of the present disclosure may also be prepared from a corresponding optically pure intermediate or by resolution, such as by HPLC of the corresponding racemate using a suitable chiral support or by fractional crystallization of the diastereomeric salts formed by reaction of the corresponding racemate with a suitable optically active acid or base, as appropriate.

The present disclosure further provides a pharmaceutical composition comprising the compound of the present disclosure and a pharmaceutically acceptable carrier.

In the context of the present disclosure, the term “alkyl” should be understood to refer to a straight, branched or where possible, cyclo hydrocarbon chain, containing the indicated number of carbon atoms, wherein all the bonds connecting the atoms are sigma bonds.

In the context of the present disclosure, the term “alkenyl” should be understood to refer to a straight, branched or where possible, cyclo hydrocarbon chain, containing the indicated number of carbon atoms, wherein at least one bond between two carbons of the chain is a double (pi) bond.

In the context of the present disclosure, the term “alkynyl” should be understood to refer to a straight, branched or where possible, cyclo hydrocarbon chain, containing the indicated number of carbon atoms, wherein at least one bond connecting two carbon atoms of the chain is a triple bond.

In the context of the present disclosure, the term “halo-alkyl” should be understood to refer to a straight, branched or where possible, cyclo hydrocarbon chain, containing the indicated number of carbon atoms, wherein all the bonds connecting the atoms are sigma bonds and at least one of the hydrogen atoms on the chain is replaced by a halogen atom selected from F, Cl, Br, I.

In the context of the present disclosure, the term “heteroalkyl” should be understood to refer to a straight, branched or where possible, cyclo hydrocarbon chain, containing the indicated number of carbon atoms, wherein the chain is interrupted or terminated by at least one heteroatom (selected from O, N, S) and all the bonds connecting the atoms are sigma bonds.

In the context of the present disclosure, the term “heteroalkenyl” should be understood to refer to a straight, branched or where possible, cyclo hydrocarbon chain, containing the indicated number of carbon atoms, wherein the chain is interrupted or terminated by at least one heteroatom (selected from O, N, S) and at least one bond between two carbons of the chain is a double (pi) bond.

In the context of the present disclosure the term “heteroalkynyl” should be understood to refer to a straight, branched or where possible, cyclo hydrocarbon chain, containing the indicated number of carbon atoms, wherein the chain is interrupted or terminated by at least one heteroatom (selected from O, N, S) and at least one bond connecting two carbon atoms of the chain is a triple bond.

The subject disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include ³C and ¹⁴C.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

Each embodiment disclosed herein is contemplated as being applicable to each of the other disclosed embodiments. Thus, all combinations of the various elements described herein are within the scope of the present disclosure.

### General Synthesis of Compounds

The compounds of the present disclosure may be prepared by techniques well known in organic synthesis and familiar to a practitioner ordinarily skilled in the art. For example, the compounds may be prepared by the synthetic transformations shown in Schemes 1-4 and in the specific examples that follow. However, these may not be the only means by which to synthesize or obtain the desired compounds.

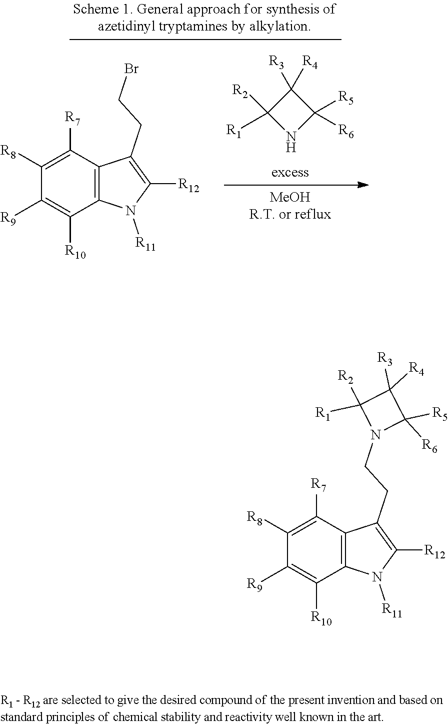

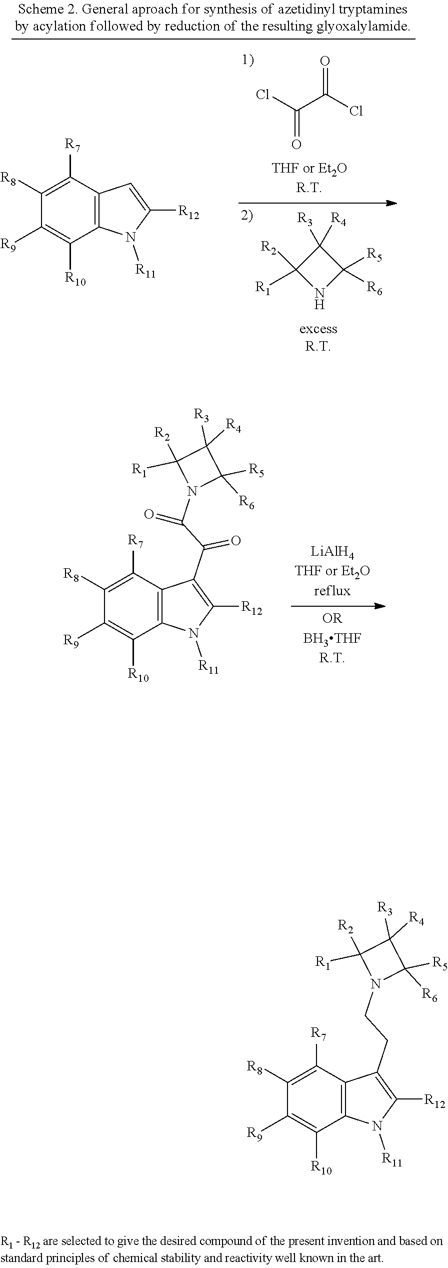

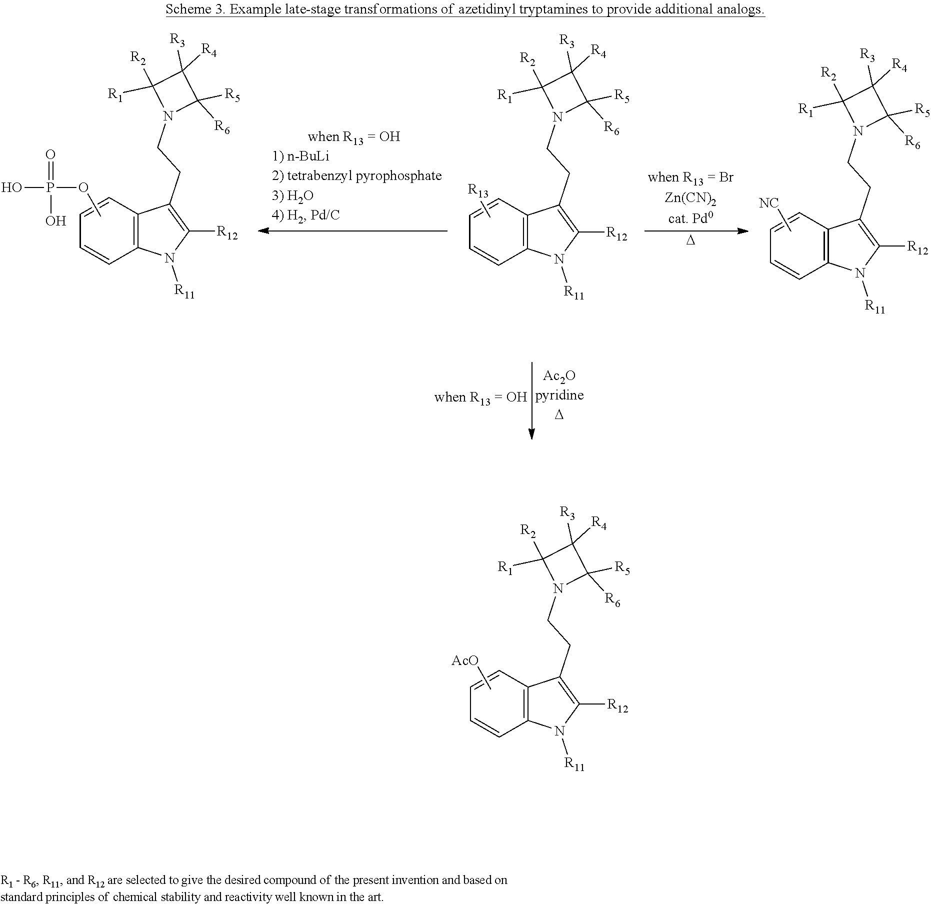

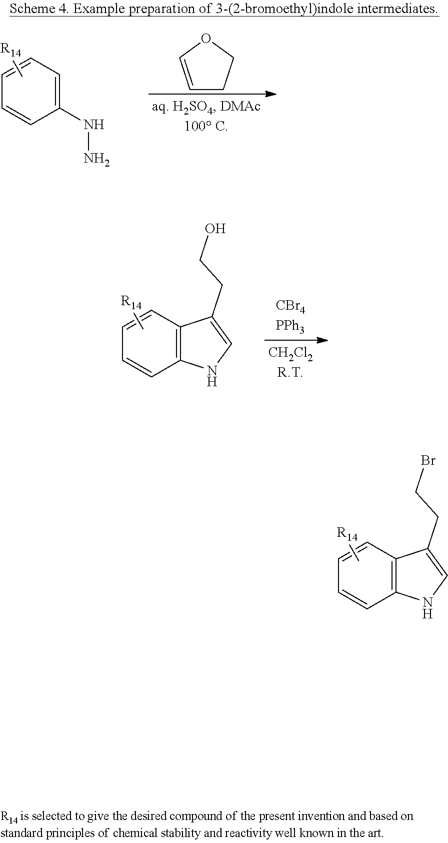

### Example 1. Preparation of Compound 1

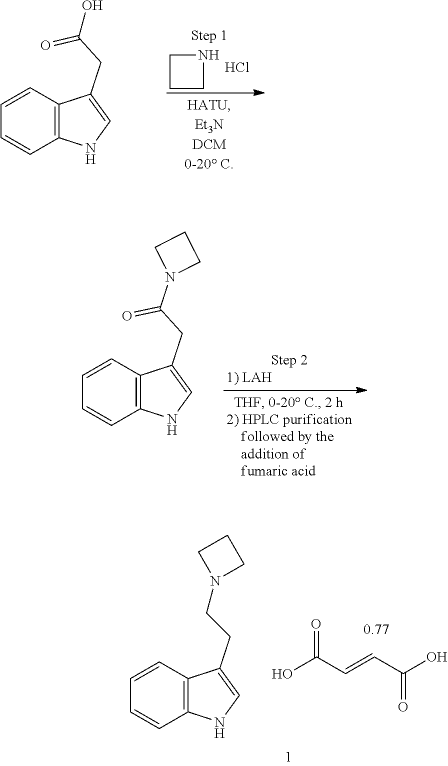

### Step 1: Preparation of 1-(azetidin-1-yl)-2-(1H-indol-3-yl)ethan-1-one

To a mixture of azetidine hydrochloride (5.61 g, 59.94 mmol, 1.5 eq) and triethylamine (12.13 g, 119.87 mmol, 16.68 mL, 3 eq) in DCM (70 mL) was added 2-(1H-indol-3-yl)acetic acid (7 g, 39.96 mmol, 1 eq) in one portion at 0° C. under N₂. To the solution was added HATU (22.79 g, 59.94 mmol, 1.5 eq) in one portion at 0° C. under N₂ and the mixture was allowed to warm to 20° C. and stirred for 2 h. Upon completion, the reaction mixture was quenched by the addition of aq. NH₄Cl (50 mL) at 20° C., and then extracted with DCM (50 mL×3). The combined organic layers were washed with brine (20 mL×3), dried over Na₂SO₄, filtered, and concentrated under reduced pressure to give a residue. The residue was purified by prep-HPLC (column=Phenomenex C18 (250*100 mm, 10 μm); mobile phase=water (NH₄HCO₃)-ACN, B %=15%-50%; 20 min run time) to afford 1-(azetidin-1-yl)-2-(1H-indol-3-yl)ethan-1-one as a white solid (3 g, 14.00 mmol, 35% yield). ¹H NMR (400 MHz, DMSO-d6) δ 10.85 (br s, 1H), 7.49 (d, J=8.0 Hz, 1H), 7.30 (d, J=8.4 Hz, 1H), 7.15 (s, 1H), 7.03 (t, J=7.6 Hz, 1H), 6.97-6.90 (m, 1H), 4.11 (t, J=7.6 Hz, 2H), 3.79 (t, J=7.6 Hz, 2H), 3.42 (s, 2H), 2.11 (quin, J=7.6 Hz, 2H).

### Step 2: Preparation of 3-(2-(azetidin-1-yl)ethyl)-1H-indole fumarate (1)

A solution of 1-(azetidin-1-yl)-2-(1H-indol-3-yl)ethan-1-one (1 g, 4.67 mmol, 1 eq) in THF (50 mL) was cooled to 0° C. Then LAH (265.68 mg, 7.00 mmol, 1.5 eq) was added and the mixture was warmed to 20° C. and stirred for 2 h. Upon completion, the mixture was cooled to 0° C., the reaction was quenched with Na₂SO₄·10H₂O until effervescence ceased, and the mixture was filtered and concentrated. The residue was purified by prep-HPLC (column=Waters Xbridge C18 (150*50 mm, 10 μm); mobile phase=water(NH₄HCO₃)-ACN, B %=1%-40%; 10 min run time) to provide a solution of 3-(2-(azetidin-1-yl)ethyl)-1H-indole in a mixture of water (800 mL) and MeCN (50 mL). To this solution was added a solution of fumaric acid (231.82 mg, 2.00 mmol) in MeCN (2 mL) in one portion at 20° C. under N₂. The solution was then lyophilized to afford 3-(2-(azetidin-1-yl)ethyl)-1H-indole fumarate (1) as a brown solid (550 mg, 1.90 mmol, 41% yield, 1:fumarate=1:0.77). ¹H NMR (400 MHz, DMSO-d6) δ 10.86 (br s, 1H), 7.53 (d, J=7.6 Hz, 1H), 7.34 (d, J=8.0 Hz, 1H), 7.15 (d, J=2.4 Hz, 11H), 7.07 (dt, J=1.2, 7.6 Hz, 1H), 6.98 (dt, 1=1.2, 7.6 Hz, 1H), 6.48 (s, 1.54H=fumarate, 0.77 mol eq), 3.46 (t, J=7.6 Hz, 4H), 2.93-2.86 (m, 2H), 2.78-2.71 (m, 2H), 2.15-2.04 (m, 2H); ¹³C NMR (101 MHz, DMSO-d6) δ 168.56, 136.68, 135.77, 127.48, 123.32, 121.41, 118.72, 111.88, 111.54, 58.16, 54.27, 22.38, 17.21; LCMS (R_{T}=1.334 min, MS calc.: 200.13, [M+H]⁺=201.1); qNMR=89%.

### Unsuccessful Methods of Work-Up and Purification for Compound 1:

Multiple alternative methods of working up the LAH reduction and purifying the resulting product 1 resulted in partial or total decomposition and did not provide pure product. These unsuccessful workup/purification methods are summarized below.

### Method 1:

Work-up: Upon completion, H₂O and 30% aq. NaOH were added to the mixture, and the mixture was filtered and concentrated.

Purification: The residue was purified by Prep-HPLC (column=Waters Xbridge BEH C18 (100*25 mm, 5 μm): mobile phase=water(NH₄HCO₃)-ACN, B %=2%-40%; 10 min run time). After lyophilization of the eluate, ¹H NMR showed that the product was impure.

### Method 2:

Work-up: Upon completion, the mixture was cooled to 0° C., the reaction was quenched with Na₂SO₄·10H₂O, and the mixture was filtered and concentrated.

Purification: The residue was purified by Prep-HPLC (HCl) (column=Phenomenex luna C18 (80*40 mm, 3 μm); mobile phase=water(HCl)-ACN, B %=1%-25%; 7 min run time). After lyophilization of the eluate, LCMS and ¹H NMR showed that the product was impure.

### Method 3:

Work-up: Same as Method 2.

Purification: The residue was purified by Prep-HPLC (column=Phenomenex luna C18 (250*70 mm, 15 μm); mobile phase=water(FA)-ACN, B %=1%-30%; 20 min run time). After lyophilization of the eluate, LCMS and ¹H NMR showed that the product was impure.

### Method 4:

Work-up: Same as Method 2.

Purification: The residue was purified by Prep-HPLC (column=Phenomenex C18 (250*50 mm, 10 μm); mobile phase=water(NH₃H₂O+NH₄HCO₃)-ACN, B %=3%-33%; 20 min run time). After lyophilization of the eluate, the residue was triturated with MTBE and the supernatant was removed, but LCMS and ¹H NMR of the remaining solid showed that the product was impure.

### Method 5:

Work-up: Same as Method 2.

Purification: The residue was purified by prep-TLC (DCM/MeOH=5:1) and column chromatography (DCM/MeOH=100/1 to 1:1), but LCMS showed that the product remained impure.

### Method 6:

Work-up: Same as Method 2.

Purification: The residue was purified by prep-HPLC (column=Phenomenex C18 (250*50 mm, 10 μm); mobile phase=water(0.05% ammonium hydroxide)-ACN, B %=5%-40%; 20 min run time). After lyophilization of the eluate, the product was impure. Then fumaric acid (˜0.5 eq) in a mixture of H₂O and ACN was added, the mixture was lyophilized again, and the residue was triturated with ether, and the supernatant was removed. However, LCMS and ¹H NMR showed that the product remained impure.

### Example 2. Preparation of Compound 2

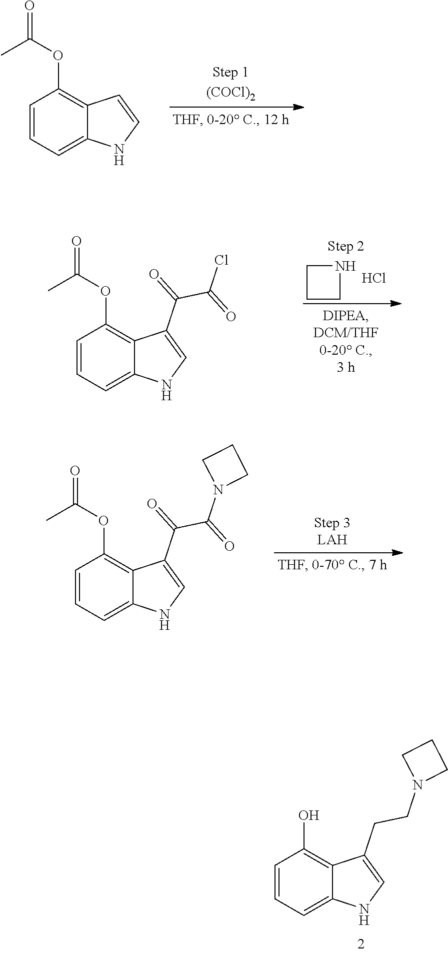

Step 1: Preparation of 3-(2-chloro-2-oxoacetyl)-1H-indol-4-yl acetate. To a solution of 1H-indol-4-yl acetate (25 g, 142.71 mmol, 1 eq) in THF (250 mL) at 0° C. was added (COCl)₂ (27.17 g, 214.06 mmol, 18.74 mL, 1.5 eq). The mixture was then allowed to warm to 20° C. and stirred for 12 h. Upon completion, the reaction mixture was concentrated to afford 3-(2-chloro-2-oxo-acetyl)-1H-indol-4-yl acetate as a yellow solid (37.91 g, 142.71 mmol, 100% yield).

Step 2: 3-(2-(azetidin-1-yl)-2-oxoacetyl)-1H-indol-4-yl acetate. To a solution of azetidine hydrochloride (19.22 g, 205.48 mmol, 1.5 eq) in DCM (100 mL) was added DIPEA (70.82 g, 547.94 mmol, 95.44 mL, 4 eq) and the mixture was stirred at 20° C. for 30 min. At this time, the mixture was cooled to 0° C., 3-(2-chloro-2-oxo-acetyl)-1H-indol-4-yl acetate (36.39 g, 136.99 mmol, 1 eq) in THF (100 mL) was added, and the mixture was allowed to warm to 20° C. and stirred for 3 h. Upon completion, aq. NH₄Cl (100 mL) was added and the mixture was stirred for 5 min. The aqueous phase was extracted with DCM (50 mL×3) and the combined organic phase was dried with anhydrous Na₂SO₄, filtered, and concentrated under vacuum. The residue was purified by column chromatography (SiO₂, petroleum ether/ethyl acetate=15/I to 0/1) to afford 3-(2-(azetidin-1-yl)-2-oxoacetyl)-1H-indol-4-yl acetate as a yellow solid (26 g, 90.82 mmol, 66% yield). ¹H NMR (400 MHz, DMSO-d₆) δ 12.47 (br s, 1H), 8.42 (s, 1H), 7.48-7.39 (m, 1H), 7.28 (t, J=7.9 Hz, 1H), 6.90 (d, J=7.3 Hz, 1H), 4.16 (t, J=7.7 Hz, 2H), 4.04 (t, J=7.8 Hz, 2H), 2.34 (s, 3H), 2.27 (pentet, J=7.8 Hz, 2H).

Step 3: Preparation of 3-(2-(azetidin-1-yl)ethyl)-1H-indol-4-ol (2). To a solution of 3-(2-(azetidin-1-yl)-2-oxoacetyl)-1H-indol-4-yl acetate (4 g, 13.97 mmol, 1 eq) in THF (150 mL) was added LAH (5.30 g, 139.72 mmol, 10 eq) at 0° C. The mixture was then heated at 70° C. for 7 h. Upon completion, the mixture was cooled to 0° C., quenched with H₂O (5.3 mL), and the mixture was filtered and concentrated under vacuum. The residue was purified by column chromatography (SiO₂, DCM/MeOH=10/1 to 0/1) to give 3-(2-(azetidin-1-yl)ethyl)-1H-indol-4-ol (2) as a pale-yellow solid (1.1 g, 5.09 mmol, 36% yield). ¹H NMR (400 MHz, DMSO-d6) δ 11.11 (br s, 1H), 10.57 (br s, 1H), 6.89 (d, J=2.4 Hz, 1H), 6.84-6.70 (m, 2H), 6.28 (dd, J=0.8, 7.2 Hz, 1H), 3.19 (t, J=7.2 Hz, 4H), 2.77-2.61 (m, 4H), 2.06-1.93 (m, 2H); ¹³C NMR (101 MHz, DMSO-d6) δ 152.37, 139.27, 122.33, 121.70, 117.36, 113.05, 104.11, 103.13, 61.83, 54.93, 25.13, 17.46; LCMS (R_{T}=2.021 min, MS calc.: 216.13, [M+H]⁺=217.1); qNMR=93%.

Note: The product 2 is unstable and should be stored frozen, protected from light, and under inert gas. If stored properly, stability has been confirmed by qNMR for at least 1 month.

Preparation of 3-(2-(azetidin-1-yl)ethyl)-1H-indol-4-ol fumarate (2 fumarate). To a solution of 3-(2-(azetidin-1-yl)ethyl)-1H-indol-4-ol (2, 100 mg, 462.37 μmol, 1 eq) in MeCN (5 mL) was added a solution of fumaric acid (37.57 mg, 323.66 μmol, 0.7 eq) in H₂O (20 mL) and MeCN (2 mL) in one portion at 20° C. under N₂ and the mixture was lyophilized to provide 3-(2-(azetidin-1-yl)ethyl)-1H-indol-4-ol fumarate (2 fumarate) as a yellow solid (125 mg, 2:fumarate=1:0.65). ¹H NMR (400 MHz, DMSO-d₆) δ 10.69 (s, 1H), 6.94 (d, J=2.0 Hz, 1H), 6.86-6.72 (m, 2H), 6.51 (s, 1H), 6.33 (dd, J=0.8, 7.2 Hz, 1H), 3.69 (t, J=7.6 Hz, 4H), 3.10 (br d, J=7.6 Hz, 2H), 2.88 (s, 2H), 2.19 (br t, J=7.6 Hz, 2H); qNMR=86%.

### Unsuccessful Methods of Work-Up and Purification for Compound 2:

Multiple alternative methods of working up the LAH reduction and purifying the resulting product 2 resulted in partial or total decomposition and did not provide pure product. These unsuccessful workup/purification methods are summarized below.

### Method 1:

Work-up: Upon completion, H₂O and 30% aq. NaOH were added to the mixture, and the resulting slurry was filtered and concentrated.

Purification: The residue was purified by Prep-HPLC (column=Welch Xtimate C18 (250*70 mm, 10 μm); mobile phase=water(NH₄HCO₃)-ACN, B %=5%-35%: 20 min run time). After lyophilization of the eluate, LCMS and ¹H NMR appeared to show that the resulting product was pure, but qNMR indicated an assay of only 62%, suggesting the presence of polymeric or insoluble impurities not visible by LCMS or NMR.

### Method 2:

Work-up: Upon completion, H₂O and 30% aq. NaOH were added to the mixture, the resulting slurry was filtered, fumaric acid (˜0.5-1 eq) was added to the filtrate, and the filtrate was concentrated.

Purification: The residue was then purified by prep-HPLC (column=Waters Xbridge Prep OBD C18 (150*40 mm*10 μm); mobile phase=water-ACN, B %=0%-30%; 20 min run time), but the product decomposed during this purification attempt.

### Method 3:

Work-up: Same as Method 1.

Purification: The residue was purified by Prep-HPLC (column=Welch Xtimate C18 (250*70 mm, 10 μm); mobile phase=water(NH₄HCO₃)-ACN, B %=5%-35%, 20 min) to provide the product in a solution of H₂O and CH₃CN. To this solution was added a solution of fumaric acid (˜0.5-1 eq) in H₂O, and the mixture was lyophilized to provide the product, which appeared pure by LCMS and ¹H NMR, but qNMR was not performed to confirm given the poor result with Method 1.

### Example 3. Preparation of Compound 3

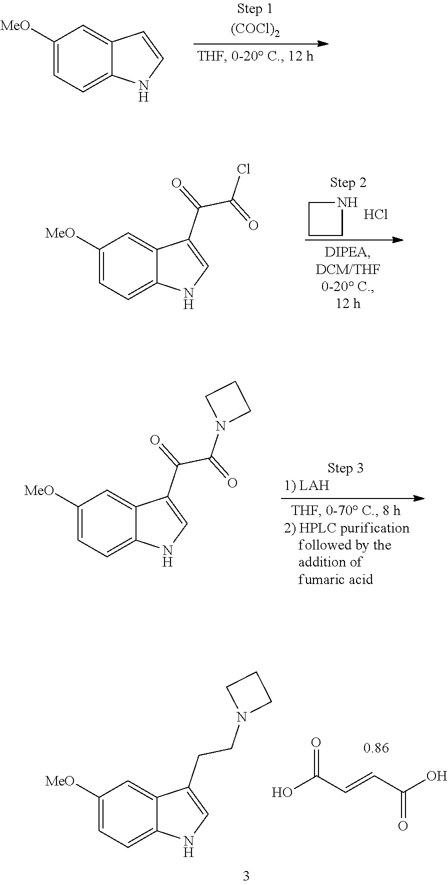

### Step 1: Preparation of 2-(5-methoxy-1H-indol-3-yl)-2-oxoacetyl chloride

To a solution of 5-methoxy-1H-indole (5 g, 33.97 mmol, 1 eq) in THF (50 mL) at 0° C. was added (COCl)₂ (6.47 g, 50.96 mmol, 4.46 mL, 1.5 eq) dropwise. The mixture was then allowed to warm to 20° C. and stirred for 12 h. TLC (PE: EA=3:1, Rf product=0.1) showed that the reaction worked well. The mixture was concentrated to afford crude 2-(5-methoxy-1H-indol-3-yl)-2-oxoacetyl chloride as a brown solid (8 g), which was used directly in the next step without further purification.

### Step 2: Preparation of 1-(azetidin-1-yl)-2-(5-methoxy-1H-indol-3-yl)ethane-1,2-dione

To a solution of azetidine hydrochloride (4.72 g, 50.50 mmol, 1.5 eq) in DCM (50 mL) was added DIPEA (17.40 g, 134.66 mmol, 23.46 mL, 4 eq) and the mixture was stirred at 20° C. for 0.5 h. At this time, the solution was cooled to 0° C., 2-(5-methoxy-1H-indol-3-yl)-2-oxoacetyl chloride (8 g, 33.66 mmol, 1 eq) in THF (100 mL) was added, and the mixture was allowed to warm to 20° C. and stirred for 12 h. TLC (PE:EA=0:1, Rf product=0.26) showed that the reaction was completed. The reaction mixture was quenched by the addition of sat. aq. NH₄Cl (10 mL) at 20° C., and then extracted with DCM (10 mL*3). The combined organic layers were washed with brine (10 mL*3), dried over Na₂SO₄, filtered, and concentrated under reduced pressure to give a residue. The crude product was purified by re-crystallization from PE (50 mL) and DCM (20 ml) at 20° C. to provide 1-(azetidin-1-yl)-2-(5-methoxy-1H-indol-3-yl)ethane-1,2-dione as a white solid (4.2 g, 16.26 mmol, 48% yield over 2 steps). ¹H NMR (400 MHz, DMSO-d6) δ 12.13 (br s, 1H), 8.42 (br s, 1H), 7.68 (br s, 1H), 7.42 (br d, J=8.7 Hz, 1H), 6.88 (br d, J=7.8 Hz, 1H), 4.33 (br t, J=7.3 Hz, 2H), 4.05 (br t, J=7.4 Hz, 2H), 3.79 (s, 3H), 2.38-2.19 (m, 2H); LCMS (R_{T}=1.226 min, MS calc.: 258.10, [M+H]⁺=259.1); Step 3: Preparation of 3-(2-(azetidin-1-yl)ethyl)-5-methoxy-1H-indole fumarate (3).

To a solution of 1-(azetidin-1-yl)-2-(5-methoxy-1H-indol-3-yl)ethane-1,2-dione (1 g, 3.87 mmol, 1 eq) in THF (50 mL) was added LAH (440.86 mg, 11.62 mmol, 3 eq) at 0° C. The mixture was then heated at 70° C. for 8 h. Upon completion, the mixture was cooled to 0° C., Na₂SO₄·10H₂O was added until effervescence ceased, and the mixture was filtered and concentrated. The residue was purified by prep-HPLC (column=Waters Xbridge Prep OBD C18 (150*40 mm, 10 μm; mobile phase=water(NH₄HCO₃)-ACN, B %=5%-35%; 8 min run time) to provide a solution of 3-(2-(azetidin-1-yl)ethyl)-5-methoxy-1H-indole in a mixture of H₂O (400 mL) and MeCN (100 mL). To this solution was added a solution of fumaric acid (˜1 eq) in MeCN (2 mL) in one portion at 20° C. under N₂. The mixture was stirred at 20° C. for 20 min and then lyophilized to provide 3-(2-(azetidin-1-yl)ethyl)-5-methoxy-1H-indole fumarate (3) as a yellow solid (300 mg, 0.91 mmol, 24% yield, 3:fumarate=1:0.86), checked by LCMS (ET47030-21-P1A1, Rt=1.543 min, M+H=231.1),

¹H NMR (400 MHz, DMSO-d₆) δ 10.71 (br s, 1H), 7.22 (d, J=8.70 Hz, 1H), 7.10 (d, J=2.03 Hz, 1H), 7.02 (d, J=2.27 Hz, 1H), 6.71 (dd, J=8.70, 2.38 Hz, 1H), 6.48 (s, 1.71H=fumarate, 0.86 mol eq), 3.76 (s, 3H), 3.53 (t, J=7.45 Hz, 4H), 2.89-2.99 (m, 2H), 2.69-2.77 (m, 2H), 2.12 (pent, J=7.51 Hz, 2H); LCMS (R_{T}=1.543 min, MS calc.: 230.14, [M+H]⁺=231.1).

### Unsuccessful Methods of Work-Up and Purification for Compound 3:

Multiple alternative methods of working up the LAH reduction and purifying the resulting product 3 resulted in partial or total decomposition and did not provide pure product. These unsuccessful workup/purification methods are summarized below.

### Method 1:

Work-up: Upon completion, H₂O and 30% aq. NaOH were added to the mixture, and the resulting slurry was filtered and concentrated.

Purification: The residue was purified by prep-HPLC (column=Waters Xbridge Prep OBD C18 (150*40 mm, 10 μm); mobile phase=water(NH₄HCO₃)-ACN, B %=1%-25%; 8 min run time). After lyophilization of the eluate, ¹H NMR showed that the product was impure.

### Method 2:

Work-up: Same as Method 1.

Purification: The residue was purified by prep-HPLC (column=Phenomenex C18 (75*30 mm, 3 μm); mobile phase=water(NH₃-H₂O+NH₄HCO₃)-ACN, B %=1%-30%; 8 min run time). After lyophilization of the eluate, ¹H NMR showed that the product was impure.

### Example 4. Preparation of Compound 4

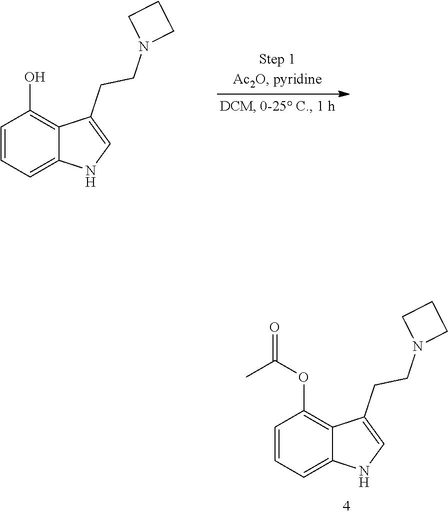 [SMILES: Oc1cccc2[nH]cc(CCN3CCC3)c12 ; CC(=O)Oc1cccc2[nH]cc(CCN3CCC3)c12]

### Step 1: Preparation of 3-(2-(azetidin-1-yl)ethyl)-1H-indol-4-yl acetate (4)

A mixture of 3-(2-(azetidin-1-yl)ethyl)-1H-indol-4-ol (2, 50 mg, 231.18 μmol, 1 eq) and pyridine (23.77 mg, 300.54 μmol, 24.26 μL, 1.3 eq) in DCM (1 mL) was cooled to 0° C. Then acetic anhydride (25.96 mg, 254.30 μmol, 23.82 μL, 1.1 eq) was added dropwise at 0° C. and the resulting mixture was stirred at 25° C. for 1 h. At this time, the solvent was removed and the residue was purified by prep-HPLC (column=Waters Xbridge BEH C18 (100*30 mm, 10 μm); mobile phase=water(NH₄HCO₃)-ACN, B %=5%-40%; 10 min run time) to provide 3-(2-(azetidin-1-yl)ethyl)-1H-indol-4-yl acetate (4) as a white solid (20 mg, 33% yield). ¹H NMR (400 MHz, DMSO-d₆) δ 11.01 (br s, 1H), 7.22 (d, J=8.0 Hz, 1H), 7.11 (d, J=1.6 Hz, 1H), 7.02 (t, J=8.0 Hz, 1H), 6.64 (d, J=7.6 Hz, 1H), 3.09 (t, J=6.8 Hz, 4H), 2.57-2.54 (m, 4H), 2.32 (s, 3H), 1.96-1.90 (m, 2H); LCMS (R_{T}=0.983 min, MS calc.: 258.14, [M+H]+=259.1). Note: The product 4 is susceptible to hydrolysis and hydrolysis was observed during analysis by LCMS and HPLC with aqueous mobile phase.

### Example 5. Metabolic Stability in Human Liver Microsomes

Disclosed compounds were tested for stability in human liver microsomes (HLM), with the results summarized in Table 1. Azetidinyl Compounds 1, 2, and 3 exhibited greater metabolic stability than their dimethyl counterparts N,N-dimethyltryptamine (DMT), psilocin, and 5-methoxy-N,N-dimethyltryptamine (5-MeO-DMT), respectively. Accordingly, the azetidinyl compounds are expected to have greater oral bioavailability than their dimethyl counterparts.

Test Compounds. Compounds 1, 2, and 3 were prepared as described above. All other compounds were commercially obtained.

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 L) were diluted with water (240 μL) and analyzed for parent compound remaining using a fit-for-purpose liquid chromatography-tandem mass spectrometry (LC-MS/MS) method.

Data Analysis. The elimination constant (kₑ₁), half-life (t_{1/2}), and intrinsic clearance (CLᵢₙₜ) were determined in a plot of ln(AUC) versus time, using linear regression analysis.

TABLE 1
Intrinsic clearance (CLᵢₙₜ) and half life (t_{1/2}) of
compounds in the presence of HLM.

| Compound | Structure | Clᵢₙₜ (μL/min/mg) | t_{1/2} (min) |
|---|---|---|---|
| DMT | 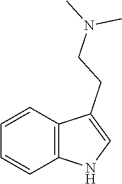 | 198.6 | 7.0 |
| Psilocin | 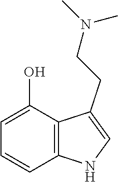 | 126.6 | 109.7 |
| 5-MeO-DMT | 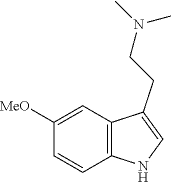 [SMILES: COc1ccc2[nH]cc(CCN(C)C)c2c1] | 101.9 | 13.6 |
| 1 | 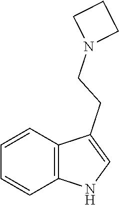 [SMILES: c1ccc2c(CCN3CCC3)c[nH]c2c1] | 12.4 | 111.8 |
| 2 | 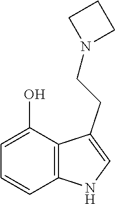 [SMILES: Oc1cccc2[nH]cc(CCN3CCC3)c12] | <9.6 | >145 |
| 3 | 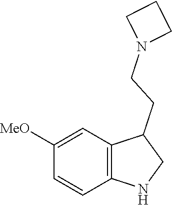 [SMILES: COc1ccc2c(c1)C(CCN1CCC1)CN2] | <9.6 | >145 |

### Example 6. Stability in the Presence of Monoamine Oxidases

Disclosed compounds were tested for stability in the presence of monoamine oxidase A and B (MAO-A and MAO-B) in human liver mitochondria preparations, with the results summarized in Table 2. Azetidinyl Compounds 1 and 3 exhibited greater stability than their dimethyl counterparts N,N-dimethyltryptamine (DMT) and 5-methoxy-N,N-dimethyltryptamine (5-MeO-DMT), respectively. Accordingly, the azetidinyl compounds are expected to experience reduced brain metabolism compared to their dimethyl counterparts. Azetidinyl Compound 2 exhibited similar stability to its dimethyl counterpart psilocin, which is already stable in this preparation.

Test Compounds. Compounds 1, 2, and 3 were prepared as described above. All other compounds were commercially obtained.

Liver Mitochondria Incubations. Human liver mitochondria (Xenotech H0610.M) were used. Mitochondrial incubations were carried out in multi-well plates. Liver mitochondrial incubation medium consisted of PBS (100 mM, pH 7.4) with 0.30 mg of liver mitochondrial protein per mL. Test compounds (1 μM, final solvent concentration 1.0%) were incubated with liver mitochondrial protein at 37° C. with constant shaking (total reaction volume 100 μL per well). Six time points over 60 minutes were analyzed. At each time point, reactions were stopped by adding 300 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (100 μL) were diluted with 5% trichloroacetic acid in water (300 μL) and analyzed for parent compound remaining using a fit-for-purpose liquid chromatography-tandem mass spectrometry (LC-MS/MS) method.

Data Analysis. The elimination constant (kₑ₁), half-life (t_{1/2}), and intrinsic clearance (CLᵢₙₜ) were determined in a plot of ln(AUC) versus time, using linear regression analysis.

TABLE 2
Intrinsic clearance (CLᵢₙₜ), half-life (t_{1/2}), and
percent remaining of compounds in the presence of
monoamine oxidases (human mitochondrial preparation).

| | t_{1/2} | CL_{int(MAO)} | Remaining |
|---|---|---|---|
| Compound | (min) | (μL/min/mg) | (t = 60 min) |
| DMT | 17.1 | 134.9 | 8.9% |
| Psilocin | >145 | <15.9 | 112.1% |
| 5-MeO-DMT | 52.9 | 43.6 | 47.1% |
| 1 | >145 | <16.0 | 85.20% |
| 2 | >145 | <16.0 | 88.00% |
| 3 | >145 | <16.0 | 106.00% |

### Example 7. Stability in Mouse Brain Homogenate

Disclosed compounds were tested for stability in mouse brain homogenate (Table 3). Azetidinyl Compounds 1, 2, and 3 all exhibited good stability under the conditions of the experiment and were much more stable than N,N-dimethyltryptamine (DMT).

Test Compounds. Compounds 1, 2, and 3 were prepared as described above. All other compounds were commercially obtained.

Brain Homogenate Stability. Frozen mouse brain homogenate (pooled from male CD-1 mice, BioreclamationIVT, MSE00BRAINMZA) was thawed in a water bath at 37° C. immediately prior to use. Positive controls and test compounds (final concentration in incubation medium=1 μM for test compounds and 2 μM for controls, all with 2% DMSO) were incubated in duplicate for each time point (0, 10, 30, 60, and 120 min) in the mouse brain homogenate at a total reaction volume of 100 μL at 37° C. At the end of each incubation period, reactions were immediately quenched with 400 μL of acetonitrile containing internal standard (200 ng/mL tolbutamine and 200 ng/mL labetalol) and mixed thoroughly. Plates were then sealed, shaken for 20 min, and centrifuged at 4,000 rpm and 4° C. for 20 min. Aliquots of 50 μL of each supernatant were diluted into 100 uL of water and the mixtures were then shaken again for 10 min. The resulting mixtures were analyzed for parent compound remaining using a fit-for-purpose LC-MS/MS method.

TABLE 3
Stability of compounds in mouse brain homogenate.

| | % Compound Remaining | t_{1/2} |
|---|---|---|
| Compound | (after 120 min) | (min) |
| DMT | 0.0 | 6.4 |
| psilocin | 97.6 | >289.1 |
| 5-MeO-DMT | 91.2 | >289.1 |
| 1 | 111.3 | >289.1 |
| 2 | 74.2 | >289.1 |
| 3 | 103.1 | >289.1 |

### Example Stability in Rat Brain Homogenate

Disclosed compounds were tested for stability in rat brain homogenate (Table 4). Azetidinyl Compounds 1, 2, and 3 all exhibited good stability under the conditions of the experiment and were more stable than their dimethyl counterparts N,N-dimethyltryptamine (DMT), psilocin, and 5-methoxy-N,N-dimethyltryptamine (5-MeO-DMT), respectively.

Test Compounds. Compounds 1, 2, and 3 were prepared as described above. All other compounds were commercially obtained.

Brain Homogenate Stability. Frozen rat brain homogenate (pooled from male Sprague Dawley rats, BioreclamationIVT, RAT00BRAINMZA) was thawed in a water bath at 37° C. immediately prior to use. Positive controls and test compounds (final concentration in incubation medium=1 μM for test compounds and 2 μM for controls, all with 2% DMSO) were incubated in duplicate for each time point (0, 10, 30, 60, and 120 min) in the rat brain homogenate at a total reaction volume of 100 μL at 37° C. At the end of each incubation period, reactions were immediately quenched with 400 μL of acetonitrile containing internal standard (200 ng/mL tolbutamine and 200 ng/mL labetalol) and mixed thoroughly. Plates were then sealed, shaken for 20 min, and centrifuged at 4,000 rpm and 4° C. for 20 min. Aliquots of 50 μL of each supernatant were diluted into 100 uL of water and the mixtures were then shaken again for 10 min. The resulting mixtures were analyzed for parent compound remaining using a fit-for-purpose LC-MS/MS method.

TABLE 4
Stability of compounds in rat brain homogenate.

| | % Compound Remaining | t_{1/2} |
|---|---|---|
| Compound | (after 120 min) | (min) |
| DMT | 0.0 | 5.1 |
| Psilocin | 46.1 | 114.8 |
| 5-MeO-DMT | 0.3 | 14.4 |
| 1 | 91.9 | >289.1 |
| 2 | 56.8 | 174.5 |
| 3 | 98 | >289.1 |

### Example 9. Functional Activity at Serotonin Receptors

Disclosed compounds were tested for agonist activity at several serotonin receptor subtypes (5-HT2A, 2-HT2B, 5-HT2C, and 5-HT1A) using Ca²⁺ flux functional assays, with the results summarized in Table 5. All compounds exhibited potent agonist activity at 5-HT2A, suggestive of potential hallucinogenic activity as well as possible therapeutic effects. However, azetidinyl compounds generally exhibited much greater potency at 5-HT1A compared to closely related compounds. For example, Compound 1 was >50-fold more potent at this receptor than its dimethyl and methylethyl counterparts, N,N-dimethyltryptamine (DMT) and N-methyl-N-ethyltryptamine (MET; N-ethyl-2-(1H-indol-3-yl)-N-methylethan-1-amine), respectively. Similarly, Compound 2 was >5-fold more potent at 5-HT1A than its dimethyl and methylethyl counterparts, psilocin and 4-hydroxy-N-methyl-N-ethyltryptamine (4-HO-MET; 3-(2-(ethyl(methyl)amino)ethyl)-1H-indol-4-ol), respectively. Lastly, Compound 3 was >10-fold more potent at 5-HT1A than its dimethyl counterpart 5-methoxy-N,N-dimethyltryptamine (5-MeO-DMT). These increases in 5-HT1A potency for the azetidinyl compounds 1, 2, and 3 were coupled with similar or slightly reduced potency at 5-HT2A, meaning that there was a relative reduction in 5-HT2A selectivity over 5-HT1A for these compounds compared to their dimethyl and methylethyl counterparts. Since 5-HT1A agonists are known to have anxiolytic and antidepressant effects, increased activity at this target is expected to enhance the therapeutic activity of the azetidinyl compounds for treatment of mood disorders.

Test Compounds. Compounds 1, 2, and 3 were prepared as described above. All other compounds were commercially obtained.

Functional Assays at 5-HT2A, 5-HT2B, and 5-HT1A. Agonist activity at 5-HT2A, 5-HT2B, and 5-HT1A receptors was determined using a FLIPR Ca²⁺ flux assay at WuXi AppTec (Hong Kong) Limited according to their standard protocols. Briefly, stably transfected cells expressing the receptor of interest (HEK293 for 5-HT2A and 5-HT2B; CHO cells for 5-HT1A) were grown and plated in a 384 well plate and incubated at 37° C. and 5% CO₂ overnight. A solution of 250 mM probenecid in 1 mL FLIPR assay buffer was prepared fresh. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

Functional Assays at 5-HT2C. Agonist activity at 5-HT2C was determined using a FLIPR Ca²⁺ flux assay at Eurofins DiscoverX (Fremont, CA) according to their standard protocols. Briefly, stably transfected cells expressing the human 5-HT2C receptor were grown and plated in a 384 well plate and incubated at 37° C. and 5% CO₂ overnight. Assays were performed in 1× Dye Loading Buffer consisting of 1× Dye, 1× Additive A, and 2.5 mM Probenecid in HBSS/20 mM Hepes. Probenecid was prepared fresh. Cells were loaded with dye prior to testing and incubated at 37° C. for 30-60 minutes. After dye loading, cells were removed from the incubator and 10 μL HBSS/20 mM Hepes was added. 3× vehicle was included in the assay buffer. Cells were incubated for 30 mins at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

TABLE 5
Agonist activity of compounds at select serotonin receptors in Ca²⁺ flux functional
assays.

| | | 5-HT2A | | 5-HT2B | | 5-HT2C | | 5-HT1A |
|---|---|---|---|---|---|---|---|---|
| | 5-HT2A | % Act @ | 5-HT2B | % Act @ | 5-HT2C | % Act @ | 5-HT1A | % Act @ |
| | EC₅₀ | Max | EC₅₀ | Max | EC₅₀ | Max | EC₅₀ | Max |
| Compound | (nM) | Dose | (nM) | Dose | (nM) | Dose | (nM) | Dose |
| 1 | 90.4 | 73.2 | 917 | 22.6 | NT | NT | 745 | 135 |
| 2 | 11.7 | 93.6 | >10,000 | 3.83 | 279 | 48.4 | 1,030 | 102 |
| 3 | 7.15 | 99.3 | 274 | 35.0 | NT | NT | 21.1 | 112 |
| DMT* | 22.2 | 93.4 | >31,600 | 10.4 | 31.3 | 92.0 | >100,000 | 68.1 |
| MET | 17.1 | 36.2 | >100,000 | 22.5 | 28.3 | 89.7 | 89,400 | 73.6 |
| psilocin* | 6.50 | 95.6 | >31,600 | 1.44 | 30.3 | 95.1 | 5,260 | 66.4 |
| 4-HO—MET | 15.1 | 103 | >10,000 | 1.09 | NT | NT | 14,700 | 73.0 |
| 5-MeO—DMT* | 1.76 | 106 | 36.7 | 17.6 | 10.1 | 89.8 | 280 | 100 |

*Values are the average of 2 or more independent experiments.

### Example 10. Effects on the Head Twitch Response (HTR) in Mice

Disclosed compounds were tested for their ability to induce a head twitch response (HTR) in mice, with the results summarized in Table 6. The maximal effect of the disclosed azetidinyl compounds (<10 head twitches/20 min) was much less than that of a prototype 5-HT2A agonist 4-iodo-2,5-dimethoxyamphetamine (DOI) (35.6 head twitches/20 min) and a prototype psychedelic tryptamine, 4-HO-MET (4-hydroxy-N-methyl-N-ethyltryptamine; 20.8 head twitches/20 min). This observation was consistent with the much greater potency of Compounds 1 and 2 as agonists of 5-HT1 A observed in vitro since 5-HT1 A agonism is known to suppress the maximal effect in the HTR assay.

Animals. Adult male C57BL/6 mice, aged 8 weeks (body weight 20-25 g) were used in these experiments. Animals were housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug Administration. Compounds 1 and 2 were prepared as described above. All other compounds were commercially obtained. Test compounds were used as the free base (2), the fumarate salt (4-HO-MET and 1), or the hydrochloride salt (DOI). Drugs were dissolved in a vehicle consisting of normal saline (DOI, 4-HO-MET, and 1) or a mixture of 10% DMSO, 10% Tween 80, and 1 molar equivalent of HCl in saline (2), and administered subcutaneously (SC) in a volume of 10 mL/kg. Test compounds were administered at 5 doses per compound (0.1 to 10 mg/kg, calculated based on the free base) using N=6 animals/group. The control compound DOI was administered at 1 dose (3.16 mg/kg, calculated based on the HCl salt), using N=12 animals.

Procedure. Mice were administered one dose of a test drug (or vehicle) SC and immediately placed into a small open field for behavioral observation. Animals were observed continuously for 20 mins and the number of HTRs were counted by an observer blind to the treatment condition.

Statistical analysis. The data points shown in Table 6 are the mean f standard error of the mean (SEM). Analysis was performed using GraphPad Prism 9.

TABLE 6
HTR of compounds in mice.

| | Dose | Average HTR Over | |
|---|---|---|---|
| Compound | (mg/kg) | 20-min Test (SEM) | |
| DOI | 3.16 | 35.583 | (3.372) |
| 4-HO-MET | 0.1 | 9.50 | (1.06) |
| 4-HO-MET | 0.316 | 17.00 | (1.44) |
| 4-HO-MET | 1 | 20.83 | (1.38) |
| 4-HO-MET | 3.16 | 14.17 | (3.32) |
| 4-HO-MET | 10 | 11.33 | (1.74) |
| 1 | 0.1 | 5.00 | (0.97) |
| 1 | 0.316 | 4.00 | (0.89) |
| 1 | 1 | 2.17 | (0.60) |
| 1 | 3.16 | 0.00 | (0.00) |
| 1 | 10 | 1.00 | (0.37) |
| 2 | 0.1 | 4.667 | (0.558) |
| 2 | 0.316 | 4.500 | (0.224) |
| 2 | 1 | 8.667 | (0.803) |
| 2 | 3.16 | 7.1637 | (1.138) |
| 2 | 10 | 9.000 | (0.516) |

### Example 11. Forced Swim Test in Rats

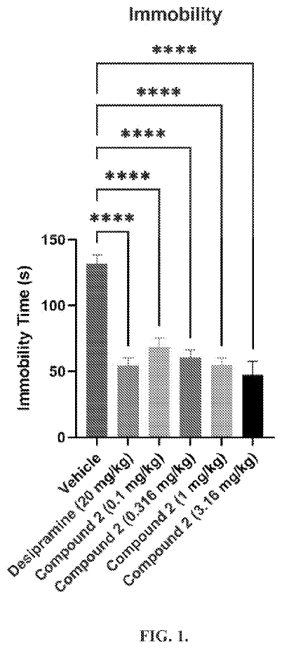

Disclosed Compound 2 induced antidepressant-like effects in the forced swim test (FST) in rats with a 23.5-h pre-treatment time (FIG. 1). Specifically, the compound reduced immobility time relative to vehicle control, indicative of an antidepressant-like effect. This effect on immobility was highly potent, with a significant effect observed even at the lowest dose tested (0.1 mg/kg). Further, the effect was observed 23.5 hours after a single compound administration, a time point at which most or all of the drug has been cleared from the systemic circulation, suggesting that Compound 2 has both rapid-acting and long-lasting antidepressant-like effects. Additionally, the compound induced significant increases in swimming (FIG. >2) behavior during the test. These effects on swimming were stronger than those induced by the control antidepressant desipramine.

Animals. Male Sprague Dawley rats, aged 8-10 weeks, were used in the experiments. Animals were housed in groups of 2 under controlled temperature (22±3° C.) and relative humidity (30-70%) conditions, with 12-hour light/dark cycles, and with ad libitum food and water. These studies were carried out in strict accordance with the requirements of the Committee for the Purpose of Control and Supervision of Experiments on Animals (CPCSEA), India. All efforts were made to minimize suffering.

Drugs and Drug Administration. Compound 2 was prepared as described above. All other compounds were commercially obtained. Test compounds, saline vehicle, and the positive control desipramine were administered subcutaneously (SC), with doses calculated based on the free base. Normal saline was used as the vehicle except for Compound 2, which was dissolved in a mixture of 10% DMSO, 10% Tween 80, and 1 molar equivalent of HCl in saline. All compounds were administered at a volume of 5 mL/kg. Test compounds and vehicle were administered 0.5 h after the start of the training swim (Swim 1) and 23.5 h before the test swim (Swim 2). Desipramine was administered 3 times, at 23.5 h, 5 h, and 1 h before the test swim (Swim 2), each time at a dose of 20 mg/kg.

Forced Swim Test (FST). Animals were randomized based on body weight, and it was ensured that inter-group variations were minimal and did not exceed ±20% of the mean body weight across the groups. Group size was N=10 per treatment, except for the vehicle and desipramine groups, which were N=20. Rats were handled for about 2 min daily for the 5 days prior to the beginning of the experimental procedure. On the first day of the experiment (i.e., Day 0), post randomization, training swim sessions (Swim 1) were conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals were dried with paper towels, placed in heated drying cages for 15 minutes, and then returned to their home cages. Animals were then administered the appropriate drug or vehicle treatment(s), as described above. For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals performed the test swim (Swim 2) for a period of 5 min but otherwise under the same conditions as Swim 1. During all swim sessions, the water was changed between each animal.

Behavioral scoring was conducted by observers who were blind to the treatment groups. Animals were continuously observed during Swim 2 and the total time spent engaging in the following behaviors was recorded: immobile, swimming, and climbing. A rat was judged to be immobile when it remained floating in the water without struggling and was making only those movements necessary to keep its head above water. A rat was judged to be swimming when it made active swimming motions, more than necessary to merely maintain its head above water (e.g., moving around in the cylinder). A rat was judged to be climbing when it made active movements with its forepaws in and out of the water, usually directed against the walls.

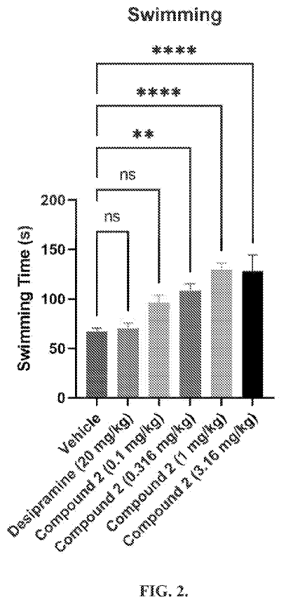

Statistical Analysis. The data points shown in FIG. 1 and FIG. 2 represent the mean+standard error of the mean (SEM). Analysis was performed using GraphPad Prism 9. Comparisons between groups were performed using the one-way analysis of variance (ANOVA), followed by Dunnett's test for comparisons to vehicle.

### Example 12. Compound 4 is a Pro-Drug of Compound 2

When administered to an animal, for example a human, the acetate ester of Compound 4 is rapidly hydrolyzed to give the phenol Compound 2 as an active metabolite. Since Compound 4 is more stable to oxidation than Compound 2, it is a useful pro-drug of Compound 2 that is more easily stored and handled. Other esters of Compound 2 (on the phenol) have similar useful properties as pro-drugs.

### Example 13. Microsomal Stability of Additional Compounds

Additional disclosed compounds are tested for stability in human liver microsomes, as described in Example 5. The compounds exhibit good stability in this preparation and are more stable than their N,N-dimethyl counterparts.

### Example 14. Stability of Additional Compounds in the Presence of Monoamine Oxidases

Additional disclosed compounds are tested to determine their stability in the presence of monoamine oxidases using liver mitochondria preparations, as described in Example 6. The compounds exhibit good stability in this preparation and are more stable than their N,N-dimethyl counterparts.

### Example 15. Agonist Activity of Additional Compounds at the 5-HT1A and 5-HT2A Receptors

Additional disclosed compounds are tested to determine their agonist activity at the 5-HT2A and 5-HT1A receptors, as described in Example 9. The compounds exhibit potent and efficacious agonist activity at both receptors and are more potent at 5-HT1 A compared to their nearest acyclic amine analogs.

### Example 16. Effects of Additional Compounds in the HTR Assay

Additional disclosed compounds are tested to determine their ability to induce a head twitch response (HTR) in mice, as described in Example 10. The compounds induce low to moderate maximal effects compared to other 5-HT2A agonists, such as DOI and 4-HO-MET.

### Example 17. Effects of Additional Compounds in the Forced Swim Test in Rats

Additional disclosed compounds are tested in the forced swim test (FST) in rats, as described in Example 11. The compounds reduce immobility in this test in a dose-dependent manner, consistent with an antidepressant-like effect.

### Example 18 Synthesis of Additional Compounds

Additional disclosed compounds may be prepared by standard methods known to those skilled in the art of organic synthesis, for example, those presented in Examples 1-4 and described elsewhere herein.

### Example 19. Preparation of Compound 5

### Method 1:

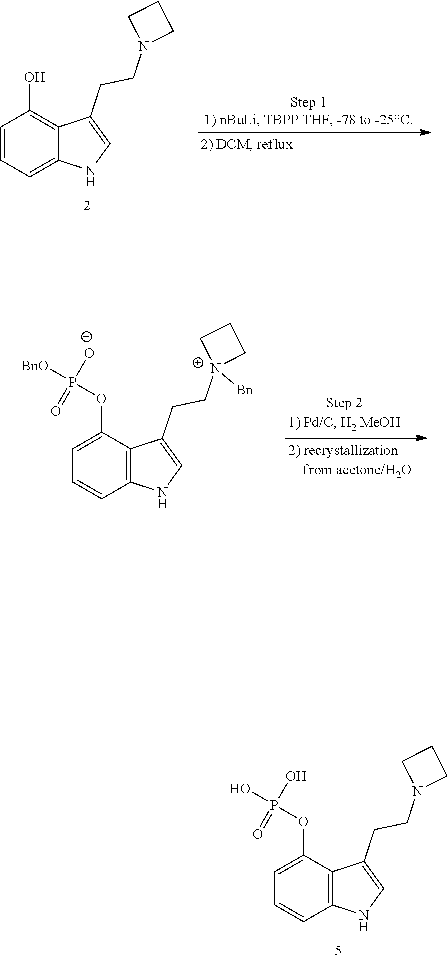 [SMILES: Oc1cccc2[nH]cc(CCN3CCC3)c12 ; O=P([O-])(OCc1ccccc1)Oc1cccc2[nH]cc(CC[N+]3(Cc4ccccc4)CCC3)c12 ; O=P(O)(O)Oc1cccc2[nH]cc(CCN3CCC3)c12]

### Step 1: Preparation of benzyl (3-(2-(1-benzylazetidin-1-ium-1-yl)ethyl)-1H-indol-4-yl) phosphate

To a mixture of 3-(2-(azetidin-1-yl)ethyl)-1H-indol-4-ol (2, 1 eq) in anhydrous THF (8.3 mL per mmol of 2) at −78° C. is added a solution of 2.5 M nBuLi in hexanes (1.2 eq) dropwise over a period of a few minutes while maintaining the internal temperature below −60° C. The reaction mixture is stirred for 10 min and then tetrabenzyl pyrophosphate (1.1 eq) is added in one portion and stirring is continued at −78° C. for 1.5 h. At this time, the cooling bath is removed and the temperature is allowed to slowly rise to −25° C. over ˜2 h. The reaction is checked for completion by LCMS. With the reaction still at −25° C., amino bound silica gel (0.5 g per mmol of 2) is added in one portion and the reaction mixture is diluted with EtOAc (10 mL per mmol of 2). The mixture is filtered through a pad of Celite and washed with EtOAc (6.7 mL per mmol of 2). The filter cake is re-slurried for 10 min with additional EtOAc (6.7 mL per mmol of 2) and again filtered. The combined filtrates are concentrated, the residue is re-dissolved in DCM (1.7 mL per mmol of 2), and the solution is heated with a heat gun to boiling for 5 min. The mixture is then allowed to cool to room temperature and then further cooled to 4° C. and held at that temperature overnight. The resulting precipitate is collected by filtration, triturated with DCM (4×1.7 mL per mmol of 2), with the supernatant removed each time, and then dried thoroughly to provide benzyl (3-(2-(1-benzylazetidin-1-ium-1-yl)ethyl)-1H-indol-4-yl) phosphate.

### Step 2: Preparation of 3-(2-(azetidin-1-yl)ethyl)-1H-indol-4-yl dihydrogen phosphate (5)

To a mixture of benzyl (3-(2-(1-benzylazetidin-1-ium-1-yl)ethyl)-1H-indol-4-yl) phosphate (1 eq) in MeOH (33.7 mL per mmol of substrate) under N₂ is added 10% Pd/C (30.9 mg per mmol of substrate) and the atmosphere is evacuated and backfilled with H₂ at 1 atm from a balloon. The reaction mixture is then stirred overnight at room temperature. The reaction is checked for completion by LCMS. The flask is then evacuated, backfilled with N₂, and the suspension is filtered through a pad of celite. The filter pad is washed with MeOH (14 mL per mmol of substrate) and the combined filtrates are concentrated to give the crude product. The crude solid is suspended in iPrOH (5.6 mL per mmol of substrate), boiled for 30 min, filtered hot (50 to 60° C.), and the collected solid is washed with acetone. This material is then suspended in 25% MeOH in iPrOH, boiled for 30 min, filtered hot, and the collected solids are washed with 25% MeOH in iPrOH. Finally, the solids are recrystallized from 30% water in acetone to give pure 3-(2-(azetidin-1-yl)ethyl)-1H-indol-4-yl dihydrogen phosphate (5). The product may be further recrystallization from 30% water in acetone or pure water to obtain material of higher purity if desired.

### Method 2:

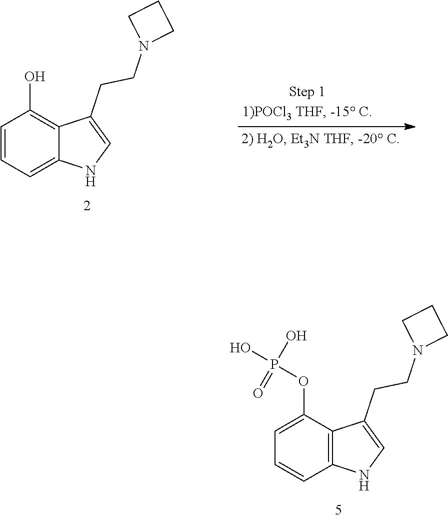 [SMILES: Oc1cccc2[nH]cc(CCN3CCC3)c12 ; O=P(O)(O)Oc1cccc2[nH]cc(CCN3CCC3)c12]

### Step 1: Preparation of 3-(2-(azetidin-1-yl)ethyl)-1H-indol-4-yl dihydrogen phosphate (5)

A slurry of 3-(2-(azetidin-1-yl)ethyl)-1H-indol-4-ol (2, 1 eq) and celite (equal weight to 2) in anhydrous THF (3.07 mL per mmol of 2) under N₂ is prepared and stirred at room temperature for at least 2 h, and the mixture is then cooled to −15° C. Separately, a solution of POCl₃ (1.5 eq) is prepared in anhydrous THF (1.36 mL per mmol POCl₃) under N₂ and cooled to −15° C. The 2/celite/THF slurry is then slowly added to the POCl₃ solution while maintaining the internal temperature between −15 and 0° C., and the resulting mixture is stirred for 1 h at −15° C. A quench solution of THF/H₂O (70:30, 2.04 mL per mmol of 2) and Et₃N (6 eq) is prepared and cooled to −20 to 0° C. The reaction mixture is then slowly added into the quench solution, maintaining the internal temperature at −20 to 0° C. Ice-cold THF (2×0.41 mL per mmol of 2) and water (0.61 mL per mmol of 2) are used to wash residues in the reaction flask into the quench mixture, maintaining the internal temperature at −20 to 0° C. The combined mixture is then stirred at −20 to 0° C. for at least 1 h. At this time, the mixture is filtered and the cake washed with water at 5 to 10° C. (2×0.41 mL per mmol of 2). The lower aqueous phase containing the product is separated, mixed with iPrOH (2.04 mL per mmol of 2), and the mixture is concentrated at <45° C. internal temperature to a volume of ca. 1.02 mL per mmol of 2 from which only water distills (with additional iPrOH added as needed to aid azeotropic distillation of water to achieve the target volume). At this point, additional water (1.02 mL per mmol 2) is added and the mixture is stirred at room temperature for at least 24 h. The resulting precipitate is collected by filtration under N₂ atmosphere, the cake is washed with cold water (2×0.41 mL per mmol of 2), and the collected solid is dried at 35-45° C. under vacuum for at least 24 h. The crude product is mixed with MeOH (10 mL per g crude product) under N₂ and stirred for at least 12 h at room temperature. The mixture is filtered under N₂ and the cake rinsed with MeOH (2×1.5 mL per g crude product) at room temperature. The collected solids are mixed with water (10 mL per g crude product) under N₂ and stirred for at least 24 h at 45-55° C. The mixture is then cooled to room temperature over ˜2 h and further stirred at that temperature for an additional 2 h. The solids are collected by filtration under N₂, washed with room temperature water (2×1 mL per g crude product), and dried at 35-45° C. under vacuum for at least 24 h to provide pure 3-(2-(azetidin-1-yl)ethyl)-1H-indol-4-yl dihydrogen phosphate (5).

### Example 20. Compound 5 is a Pro-Drug of Compound 2

When administered to an animal, for example a human, the phosphate ester of Compound 5 is rapidly hydrolyzed to give the phenol Compound 2 as an active metabolite. Since Compound 5 is more stable than Compound 2, it is a useful pro-drug of Compound 2 that is more easily stored and handled.

It should be understood that the examples and embodiments provided herein are exemplary. Those skilled in the art will envision various modifications of the examples and embodiments that are consistent with the scope of the disclosure herein. Such modifications are intended to be encompassed by the claims.

1. A compound having the general Formula I: 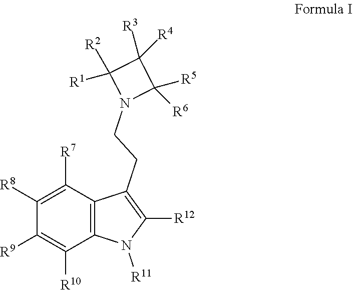
Wherein
R¹-R⁶ are each independently selected from H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, C₁-C₅ halo-alkyl;
R⁷-R¹⁰ and R¹² are each independently selected from H, F, Cl, Br, I, CF₃, SF₅, C₁-C₁₀ alkyl, C₂-C₁₀ alkenyl, C₂-C₁₀ alkynyl, C₁-C₁₀ heteroalkyl, C₂-C₁₀ heteroalkenyl, C₂-C₁₀ heteroalkynyl, C₁-C₁₀ halo-alkyl, —CN, —O—(C₁-C₁₀ alkyl), —O—(C₁-C₁₀ heteroalkyl), —S—(C₁-C₁₀ alkyl), —S—(C₁-C₁₀ heteroalkyl), —S(O)—(C₁-C₁₀ alkyl), —SO₂—(C₁-C₁₀ alkyl), OH, —CO₂H, —C(O)—NH₂, —C(O)—NH—(C₁-C₁₀ alkyl), —CO₂—(C₁-C₁₀ alkyl), —O—C(O)—(C₁-C₁₀ alkyl), —O—P(O)(OH)(OH), NH₂, —NH—(C₁-C₁₀ alkyl), —N(C₁-C₁₀ alkyl)(C₁-C₁₀ alkyl), NO₂, OCF₃; and
R¹¹ is selected from H, C₁-C₅ alkyl, C₂-C₅ alkenyl, C₂-C₅ alkynyl, C₁-C₅ heteroalkyl, C₂-C₅ heteroalkenyl, C₂-C₅ heteroalkynyl, C₁-C₅ halo-alkyl;
or a pharmaceutically acceptable salt or ester thereof.

2. A compound according to claim 1, wherein
R¹-R⁶ are each independently selected from H, Me, Et, n-Pr, i-Pr, cyclopropyl, —CHCH₂ (vinyl), —CCH (ethynyl), —CH₂CHCH₂ (allyl);
R⁷-R¹⁰ and R¹² are each independently selected from —H, —F, —Cl, —Br, —I, —CF₃, —SF₅, -Me, -Et, -n-Pr, -i-Pr, cyclopropyl, —CHCH₂ (vinyl), —CCH (ethynyl), —CH₂CHCH₂ (allyl), —CN, —OMe, —OEt, —SMe, —SEt, —OH, —OAc, —CO₂H, —C(O)—NH₂, —CO₂Me, —O—C(O)—(C₁-C₅ alkyl), —O—P(O)(OH)(OH), —NH₂, —NO₂, —OCF₃; and
R¹¹ is selected from —H, -Me, -Et, -n-Pr, -i-Pr, cyclopropyl, —CH₂CHCH₂ (allyl);
or a pharmaceutically acceptable salt or ester thereof.

3. A compound according to claim 1, wherein
R¹-R⁶ are each independently selected from —H, -Me, -Et;
R⁷-R¹⁰ and R¹² are each independently selected from —H, —F, —Cl, —Br, —I, —CF₃, —SF₅, -Me, -Et, —CN, —OMe, —SMe, —OH, —OAc, —C(O)—NH₂, —O—P(O)(OH)(OH), —NH₂, —NO₂, —OCF₃; and
R¹¹ is selected from —H, -Me, -Et;
or a pharmaceutically acceptable salt or ester thereof.

4. A compound according to claim 1, wherein
R¹-R⁶ are each independently selected from —H, -Me, -Et;
R⁷-R¹⁰ and R¹² are each independently selected from —H, —F, —Cl, —Br, —I, —CF₃, -Me, —CN, —OMe, —OH, —OAc, —C(O)—NH₂, —O—P(O)(OH)(OH), —NH₂; and
R¹¹ is —H;
or a pharmaceutically acceptable salt or ester thereof.

5. The compound according to claim 1, wherein the compound is represented by Formula (I-a): 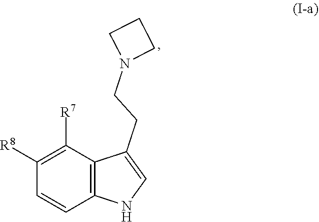
or a pharmaceutically acceptable salt thereof.

6. The compound according to claim 5, wherein R⁷ is selected from the group consisting of —H, —OH, —O—(C₁-C₁₀ alkyl), —O—C(O)—(C₁-C₁₀ alkyl), and —O—P(O)(OH)(OH).

7. The compound according to claim 6, wherein R⁷ is selected from the group consisting of —H, —OH, —OAc, and —O—P(O)(OH)(OH).

8. The compound according to claim 5, wherein R⁸ is selected from the group consisting of —H, —OH, —O—(C₁-C₁₀ alkyl), and —O—C(O)—(C₁-C₁₀ alkyl).

9. The compound according to claim 8, wherein R⁸ is selected from the group consisting of H, —OH, —OMe and —OAc.

10. A compound according to claim 1, being selected from the group consisting of: 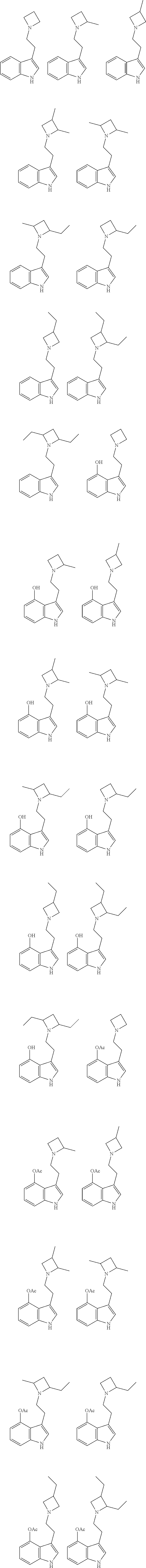 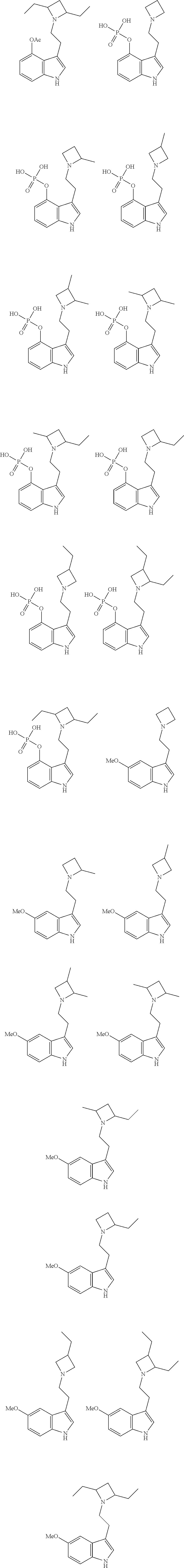
or a pharmaceutically acceptable salt or ester thereof.

11. A compound according to claim 1, being selected from the group consisting of: 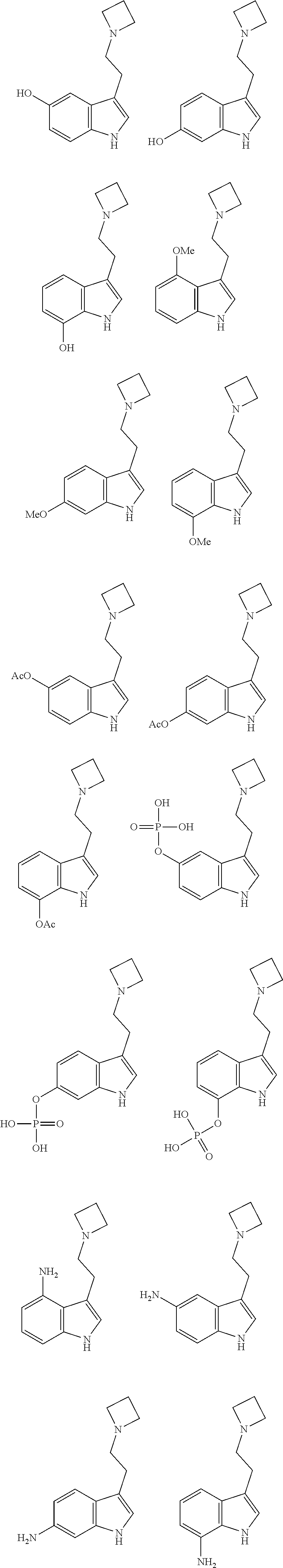
or a pharmaceutically acceptable salt or ester thereof.

12. A compound according to claim 1, being selected from the group consisting of: 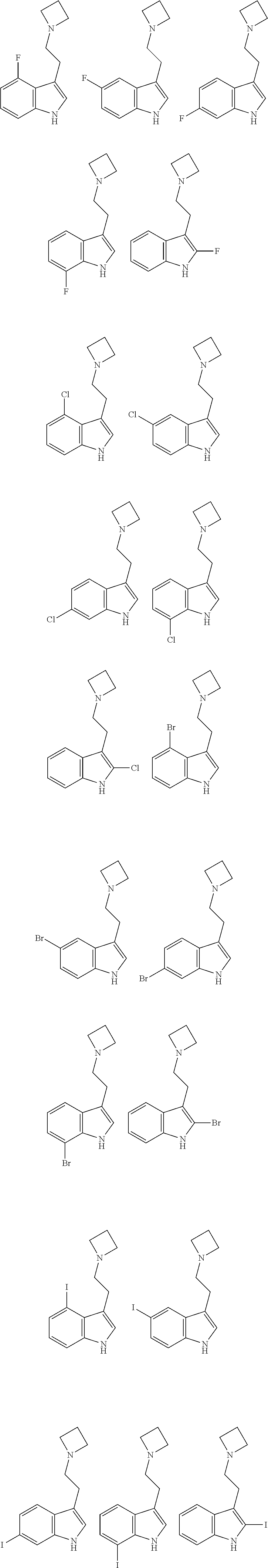 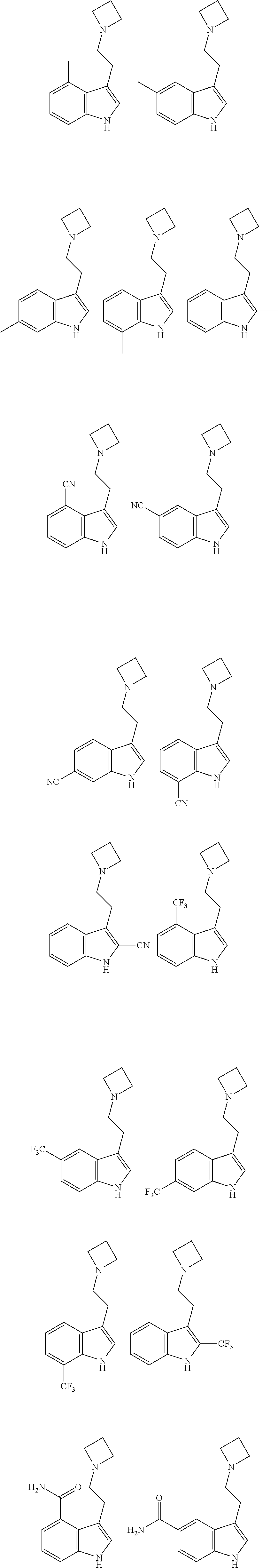
or a pharmaceutically acceptable salt thereof.

13. A compound according to claim 1, being selected from the group consisting of: 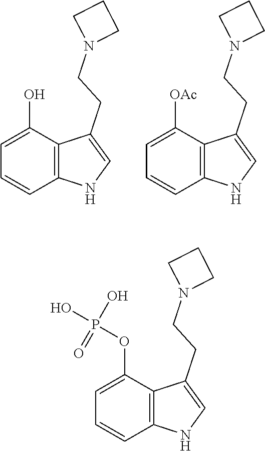
or a pharmaceutically acceptable salt or ester thereof.

14. A compound according to claim 1, having the structure: 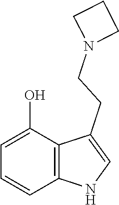 [SMILES: Oc1cccc2[nH]cc(CCN3CCC3)c12]
or a pharmaceutically acceptable salt thereof.

15. A compound according to claim 1, having the structure: 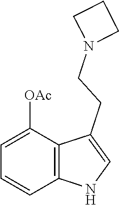 [SMILES: CC(=O)Oc1cccc2[nH]cc(CCN3CCC3)c12]
or a pharmaceutically acceptable salt thereof.

16. A compound according to claim 1, having the structure: 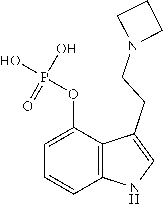 [SMILES: O=P(O)(O)Oc1cccc2[nH]cc(CCN3CCC3)c12]
or a pharmaceutically acceptable salt thereof.

17. A compound according to claim 1, having the structure: 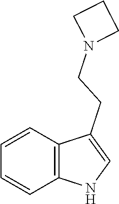 [SMILES: c1ccc2c(CCN3CCC3)c[nH]c2c1]
or a pharmaceutically acceptable salt thereof.

18. A compound according to claim 1, having the structure: 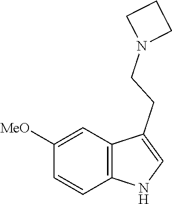 [SMILES: COc1ccc2[nH]cc(CCN3CCC3)c2c1]
or a pharmaceutically acceptable salt thereof.

19. A pharmaceutical composition comprising one or more compound of claim 1.

20. A method of treating a psychiatric disease or disorder in a patient in need thereof, said method comprising administering to said subject a composition comprising an effective amount of a compound of claim 1.

21. The method according to claim 20, wherein said psychiatric disease or disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

22. The method according to claim 20, wherein said psychiatric disease or disorder is selected from the group consisting of bipolar disorder I, bipolar disorder II, cyclothymic disorder, substance/medication-induced bipolar and related disorder, and bipolar and related disorder due to another medical condition.

23. The method according to claim 20, wherein said psychiatric disease or disorder is a substance-related disorder or substance-use disorder.

24. The method according to claim 20, wherein said psychiatric disease or disorder is selected from the group consisting of separation anxiety disorder, selective mutism, specific phobia, social anxiety disorder, panic disorder, panic attach, agoraphobia, generalized anxiety disorder, substance/medication-induced anxiety disorder, anxiety disorder due to another medical condition.

25. The method according to claim 20, wherein said psychiatric disease or disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

26. The method according to claim 20, wherein the psychiatric disorder is a neurocognitive disorder.

27. The method according to claim 20, wherein the psychiatric disease or disorder is a treatment-resistant disease or disorder.

28. The method according to claim 20, wherein the method provides improvement in at least one symptom selected from the group consisting of sadness or lethargy or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, irritability, fatigue, feelings of worthlessness or low self-esteem, strongly held negative beliefs or pessimistic thoughts about self, others or world, feelings of helplessness, inability to concentrate or distractibility, recurrent thoughts of death or suicide, feelings of guilt, memory complaints, difficulty experiencing positive feelings, feeling cut off or distant from people, hypervigilance, risk taking behavior, avoidance of thoughts about a stressful or traumatic event, pains and aches, ruminations and obsessive thoughts, compulsive behaviors, talking to people you don't know well or strangers, being center of attention, disturbing intrusive thoughts, can't get through week without drug use, guilty about drug use, problems with friends or family due to drug use, and withdrawal symptoms due to drug use.

29. A method of enhancing creativity or cognition in a subject, said method comprising administering to said subject a composition comprising an effective amount of a compound of claim 1.
